SEQUENCE LISTING (5 sequences)

SEQ ID NO: 1 - protein, 31 aa, synthetic construct
  REGION 1..31: note = Casp8p41 BH3-like peptide
DMNLLDIFIEMEKRVILGEGKLDILKRVCAQ

SEQ ID NO: 2 - protein, 23 aa, synthetic construct
  REGION 1..23: note = N-terminal control peptide
MDFSRNLYDIGEQLDSEDLASLK

SEQ ID NO: 3 - DNA, 22 bp, synthetic construct
  source 1..22: mol_type = other DNA
ttaagcctcaataaagcttgcc

SEQ ID NO: 4 - DNA, 20 bp, synthetic construct
  source 1..20: mol_type = other DNA
gttcgggcgccactgctaga

SEQ ID NO: 5 - DNA, 26 bp, synthetic construct
  source 1..26: mol_type = other DNA
ccagagtcacacaacagacgggcaca

(19) United States
(12) Patent Application Publication
(10) Pub. No.: US 2024/0374532 A1
(43) Pub. Date: Nov. 14, 2024

(54) METHODS AND MATERIALS FOR KILLING HIV INFECTED CELLS

(71) Applicant: Mayo Foundation for Medical Education and Research, Rochester, MN (US)

(72) Inventors: Andrew D. Badley, Rochester, MN (US); Amy M. Nguyen, Salt Lake City, UT (US); Scott H. Kaufmann, IV, Rochester, MN (US); Yuan-Ping Pang, Rochester, MN (US); Haiming Dai, Rochester, MN (US)

(73) Assignee: Mayo Foundation for Medical Education and Research, Rochester, MN (US)

(21) Appl. No.: 18/444,033

(22) Filed: Feb. 16, 2024

Related U.S. Application Data
(63) Continuation of application No. 17/203,594, filed on Mar. 16, 2021, now Pat. No. 11,938,098.
Application No. 17/203,594 is a continuation of application No. 15/567,855, filed on Oct. 19, 2017, now abandoned.
(60) Provisional application No. 62/149,873, filed on Apr. 20, 2015.

(51) Int. Cl. A61K 31/00; A61K 31/21; A61K 31/635; A61K 38/20; A61K 45/06; A61P 31/18

(52) U.S. Cl. CPC A61K 31/00; A61K 31/635; A61P 31/18; A61K 31/21; A61K 38/2013; A61K 45/06

(57) ABSTRACT

This document provides methods and materials involved in killing HIV infected cells (e.g., CD4 T cells). For example, methods and materials for using one or more Bcl-2 inhibitors (e.g., ABT-199) alone or in combination with one or more agents capable of reactivating HIV (e.g., latency reversing agent) to kill HIV infected cells (e.g., CD4 T cells) are provided.

### CROSS-REFERENCE TO RELATED APPLICATIONS

This application is a continuation of U.S. application Ser. No. 17/203,594, filed Mar. 16, 2021, which is a continuation U.S. application Ser. No. 15/567,855, filed Oct. 19, 2017 (Abandoned), which is a National Stage application under 35 U.S.C. § 371 of International Application No. PCT/US2016/028419, having an International Filing Date of Apr. 20, 2016, which claims priority to U.S. Application Ser. No. 62/149,873, filed on Apr. 20, 2015. The disclosure of the prior applications are considered part of the disclosure of this application, and are incorporated in its entirety into this application.

### BACKGROUND

### 1. Technical Field

This document relates to methods and materials involved in killing HIV infected cells (e.g., CD4 T cells). For example, this document provides methods and materials for using one or more Bcl-2 inhibitors (e.g., ABT-199) alone or in combination with one or more agents capable of reactivating HIV (e.g., latency reversing agent) to kill HIV infected cells (e.g., CD4 T cells).

### 2. Background Information

HIV is a retrovirus that causes the acquired immunodeficiency syndrome (AIDS), which is a medical condition where progressive failure of the immune system leads to life-threatening opportunistic infections. The HIV infection, while treatable for long periods of time, remains a largely incurable infection. On the other hand, an HIV infection was “cured” in one patient, which involved using myeloablative chemotherapy and maximally suppressive antiretroviral therapy (ART), followed by bone marrow transplantation (BMT; Hütter et al., N. Engl. J Med., 360:692-698 (2009)).

### SUMMARY

This document provides methods and materials involved in killing HIV infected cells (e.g., CD4 T cells). For example, this document provides methods and materials for using one or more Bcl-2 inhibitors (e.g., ABT-199) alone or in combination with one or more agents capable of reactivating HIV (e.g., latency reversing agent) to kill HIV infected cells (e.g., CD4 T cells). As described herein, Bcl-2 inhibitors (e.g., ABT-199) alone or in combination with one or more agents capable of reactivating HIV (e.g., latency reversing agent) can be used to kill HIV infected cells (e.g., CD4 T cells such as latently HIV infected CD4 T cells or central memory CD4 T cells).

In general, one aspect of this document features a method for killing HIV infected cells within a human infected with HIV. The method comprises, or consist essentially of, (a) administering a Bcl-2 inhibitor to the human, and (b) administering a latency reversing agent to the human. The cells can be CD4+ T cells. The Bcl-2 inhibitor can be ABT-199. The latency reversing agent can be selected from the group consisting of an HDAC inhibitor, a phorbol ester, IL-2, and a bromodomain inhibitor.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-1H. Casp8p41 binds the BH3 binding grooves of antiapoptotic Bcl-2 family members. FIG. 1A, 293T cells transfected with HA-empty vector, HA-Casp8p41, or HA-Casp8p41EK were immunoprecipitated with anti-HA-conjugated beads or agarose conjugated antibody (control IgG or Bcl-2) and immunoblotted as indicated. FIG. 1B, Binding of 12.5-200 nM GST-Bcl-2 to immobilized GST-Casp8p41 as assessed by SPR. FIG. 1C, KDs of GST-Bcl-2, GST-Bcl-xL and GST-Mcl-1 binding to GST-Casp8p41. FIG. 1D, Multiple low-mass molecular dynamics simulation-refined model of the Casp8p41 activator domain (green) binding the human Bcl-2 BH3-binding groove. FIG. 1E, Binding of 800 nM GST-Bcl-2 or GST-Bcl-2 R146A to immobilized Casp8p41. FIG. 1F, KDs determined as in panel FIG. 1E. FIG. 1G, Binding of 200 nM GST-Casp8p41 or GST-Casp8p41 Val150Glu/Leu157Lys to immobilized GST-Bcl-2. FIG. 1H, KDs determined as in panel FIG. 1G. Bars in FIG. 1C, FIG. 1F, and FIG. 1H: mean±SD from 3 independent experiments.

FIGS. 2A-2I. T_{CM} are more resistant to cell death than T_{EM}. FIG. 2A, Representative flow data demonstrating immunophenotyping gating strategy for naïve, central memory, and effector memory CD4 T cell analysis (left panel); light scatter characterization of cell viability and LIVE/DEAD staining for cell viability and death (right panel). FIG. 2B, FIG. 2C, PBMCs from 3 uninfected donors were treated overnight with agonistic anti-Fas antibody CHI 1, cycloheximide (CHX), etoposide (ETP), camptothecin (CPT), carbonyl cyanide 3-chlorophenylhydrazone (CCCP) or H₂O₂, cell death was assessed by flow cytometry using light scatter (FIG. 2B) and LIVE/DEAD stains (FIG. 2C). FIG. 2D, T_{CM} and T_{EM} were isolated from two HIV-infected donors by magnetic bead separation and gene expression measured by NGS. Depicted are differentially expressed genes associated with cell proliferation and cell death sorted by log 2 fold change. Cell proliferation genes are at the top of the figure and cell death genes below. FIG. 2E, Cytosolic extracts from T_{CM} and T_{EM} were assessed for expression of procaspase 8 (Casp8) and Bcl-2 by Western blot and densitometry. Representative of three experiments. FIG. 2F, PBMCs from 2 HIV-infected patients were assessed for intracellular Casp8p41 expression in T cell subsets, using CD3-negative cells as the negative gating control. PBMCs from five HIV-infected patients were assessed for intracellular Casp8p41 expression in CD4+ T cell subsets and in CD3 negative cells (FIG. 2G). Depicted is representative flow cytometry data showing the gating strategy to determine intracellular Casp8p41 expression in CD4⁺ T cell subsets: Naïve (TN), Central Memory (TCM), Effectory Memory (TEM), and total CD4 T cells (FIG. 2H). CD3-cells, which are not infected by HIV and thus do not express HIV protease, were used as negative gating controls. (FIG. 2I) Gene expression differences in TCM versus TEM were validated in a separate publicly available dataset of 6 uninfected donors.

FIGS. 3A-3H. Bcl-2 overexpression decreases Casp8p41- or HIV-induced cell death while increasing viral replication. FIG. 3A, Parental Jurkat T cells or Jurkat cells stably overexpressing Bcl-2 were transfected with GFP-Casp8p41 or vector control and assessed for cell death via TUNEL. FIG. 3B, Relative GFP-Casp8p41 expression in EGFP⁺ Jurkat and Jurkat/Bcl-2 cells. Cells infected with HIVIIIb or mock infected were assessed for viability by LIVE/DEAD stain (FIG. 3C), cell associated HIV DNA content (FIG. 3D), and HIV p24 production in culture supernatant (FIG. 3F), at day 9 post infection. Bars, mean±SD from 3 independent experiments. (FIG. 3H, FIG. 3E) Parental Jurkat cells or Jurkat-BCL-2 cells were transfected with GFP-Casp8p41 or GFP alone and assessed for active BAK expression in GFP positive cells, using a conformational specific antibody which detects only active BAK. Depicted are individual MFI of active BAK (FIG. 3H) and representative dot plots (FIG. 3E) from two independent experiments. (FIG. 3F) Parental Jurkat cells or Jurkat-BCL-2 cells were infected with HIVIIIb or mock infected, and assessed for HIV p24 production in culture supernatant at day 9 post infection. Depicted are mean (SD) values of three independent experiments. (FIG. 3G) Parental Jurkat T cells or Jurkat T cells stably overexpressing BCL-2 (Jurkat-BCL-2) were transfected with GFP-Casp8p41 or GFP alone and assessed for cell death via TUNEL.

FIG. 4. Bcl-2 overexpression decreases TUNEL positivity induced by Casp8p41. Depicted is representative flow data from 6 hours post-transfection of parental Jurkat cells and Jurkat+ Bcl-2 cells comparing TUNEL positivity in EGFP or EGFP-Casp8p41 expressing cells. Numbers depict the percent of GFP positive cells that are TUNEL positive.

FIG. 5. Bcl-2 overexpression increases cell viability after in vitro HIV infection. Depicted is representative flow data assessing cell viability using LIVE/DEAD staining in parental Jurkat cells and Jurkat+ Bcl-2 cells. Numbers depict percent of viable cells.

FIGS. 6A-6B. Casp8p41 activator peptide binding to Bcl-2 is reduced by Arg146Ala substitution. FIG. 6A, Binding of 800 nM of GST-Bcl-2 or GST-Bcl-2 Arg146Ala to immobilized Casp8p41 activator peptide as assessed by SPR. FIG. 6B, Dissociation constants of GST-Bcl-2 and GST-Bcl-2 R146A to Casp8p41 activator peptide, from three independent experiments. (P<0.05).

FIGS. 7A-7I. ABT-199 decreases infected cell survival, HIV replication, and cell associated HIV DNA during acute HIV infection in vitro. FIG. 7A, Jurkat T cells pretreated with DMSO or ABT-199 were transfected with EGFP-Casp8p41 or vector control and assayed for cell death by TUNEL. FIG. 7B, Primary CD4 T cells from 7 uninfected donors were infected with HIVIIIb or mock infected, treated with ABT-199 or diluent, and assayed for cell viability. FIG. 7C, Primary CD4 T cells were stimulated with phytohemagglutinin in the presence of ABT-199 or control and assayed for proliferation by CFSE staining. FIG. 7D, FIG. 7E, at day 9 post infection, HIV p24 concentrations in culture supernatant (FIG. 7D) and cell-associated HIV DNA (FIG. 7E), were measured. FIG. 7F, Primary CD4 T cells were transfected with an HIV LTR-luciferase reporter, treated with increasing concentrations of ABT-199, and stimulated with prostratin (1 μM). After overnight incubation, luciferase activities were measured. FIG. 7G-7I, Primary CD4 T cells infected with HIV in the presence or absence of ABT-263 (1 μM) were assayed for viability (FIG. 7G), supernatant p24 production (FIG. 7H), and cell associated HIV-DNA (FIG. 7I). Bars: mean±SD of 4 (A) or 3 (other panels) independent experiments.

FIG. 8. ABT-199 increases Casp8p41-induced apoptosis. Depicted are representative dot plots assessing apoptosis as measured by TUNEL staining in Jurkat cells transfected with EGFP or EGFP-Casp8p41 and treated with diluent (DMSO) or increasing concentrations of ABT-199. Numbers depict the percent of EGFP positive (or total for No DNA) cells that are TUNEL positive.

FIG. 9. ABT-199 decreases cell viability following acute HIV infection in vitro. Depicted is representative flow data assessing cell viability using LIVE/DEAD staining in primary CD4 T cells mock infected or HIV infected and treated with DMSO or ABT-199 at day 9 post-infection. Numbers depict the percent of cells that are viable.

FIGS. 10A-10E. Selective Bcl-2 inhibition reduces HIV DNA associated with cells after viral reactivation ex vivo. FIG. 10A, Primary CD4 T cells isolated by negative selection from cryopreserved PBMCs of long-term virologically suppressed HIV-infected patients (N=4) were treated for 16 hours with ABT-199 or vehicle control in the presence of tenofovir and raltegravir, then exposed to plate bound αCD3 and soluble αCD28 antibody to induce HIV reactivation. After 72 hour, cell associated HIV DNA was measured. FIG. 10B, Freshly obtained peripheral CD4 T cells from an additional 16 suppressed HIV-infected patients were treated with ABT-199 or DMSO followed by αCD3/αCD28 for 72 hours before cell associated HIV DNA was measured. Depicted is the ratio of HIV DNA in ABT-199 treated vs. diluent treated samples for each patient with measurable HIV DNA in the diluent sample (n=11). FIG. 10C, Primary CD4 T cells from HIV-infected subjects were treated with ABT-199 or diluent and assayed for intracellular Casp8p41 and active caspase 3 in the T_{CM} subset. Representative data of three independent subjects. (FIG. 10D) Primary CD4 T cells from four HIV-infected subjects were treated with venetoclax or diluent and induced to reactivate HIV using CD3/CD28, and cell death measured in HIV P24 positive or negative cells using activated caspase 3 staining. (FIG. 10E) HIV RNA was measured in cell culture supernatant from 6 of the 11 patient experiments from FIG. 10B.

FIGS. 11A-11D. Venetoclax increases Casp8p41-induced apoptosis but spares cells that do not express Casp8p41. (FIG. 11A) Jurkat T cells pretreated with increasing concentrations of venetoclax or diluent DMSO were transfected with EGFP-Casp8p41, vector control, or no DNA, and assayed for cell death by TUNEL. Data are representative of three independent experiments. (FIG. 11B) Primary CD4 T cells from seven uninfected donors were treated with venetoclax (1 μM) or diluent, and assayed for cell viability by flow cytometry over 5 days. (FIG. 11C) Primary CD4 T cells from four uninfected donors were stimulated with phytohemagglutinin (2 μg/mL) in the presence of venetoclax (1 μM) or diluent and assayed for proliferation by CFSE staining. (FIG. 11D) Primary CD4 T cells from seven ART-suppressed HIV positive patients were treated with venetoclax (1 μM) or DMSO control for 24 hours and assessed for viability by trypan blue exclusion.

FIGS. 12A-12G. Apoptotic cell death is associated with loss of protein and mRNA markers. Jurkat T cells stably expressing eGFP were treated with DMSO or CPT and assessed for cell viability by ATP content (FIG. 12A) and light scatter (FIG. 12B) over time. (FIG. 12C) Expression of eGFP was measured by flow cytometry. (FIG. 12D) Actin mRNA expression was assessed by qRT-PCR and expressed as mean Ct values normalized to baseline DMSO control. (FIG. 12E-12G) Cell death was assessed by active-caspase 3 expression (FIG. 12E), TUNEL (FIG. 12F), and LIVE/DEAD viability stain (FIG. 12G) and compared between eGFP positive and eGFP negative cells. FIG. 12A-12D values represent mean (range) of two independent experiments. FIG. 12E-12G are representative of two independent experiments.

FIGS. 13A-13B. BCL-2 antagonism as a model of “prime, shock and kill” for decreasing the HIV reservoir. (FIG. 13A) Reactivation of latent HIV does not lead to death of the reactivated cell due to inhibition of Casp8p41 by direct binding to BCL-2. Therefore, the reactivated cell survives and produces progeny virus. (FIG. 13B) Antagonism of BCL-2 activity (as with venetoclax treatment) allows Casp8p41 to bind pro-apoptotic BAK, leading to apoptotic death of the reactivated cell, and decreased total HIV DNA.

### DETAILED DESCRIPTION

This document provides methods and materials for treating HIV infections. For example, this document provides methods and materials for using one or more Bcl-2 inhibitors alone or in combination with one or more other agents to treat HIV infections. In some cases, one or more Bcl-2 inhibitors can be used to cause latently HIV infected cells to die following HIV reactivation in those latently HIV infected cells.

Any appropriate method can be used to identify a human having an HIV infection. For example, HIV blood tests can be used to identify a human having an HIV infection.

Once identified as having an HIV infection, the human can be administered ART (e.g., maximally suppressive antiretroviral therapy) to prevent or reduce the level of repopulation of the HIV reservoir and one or more Bcl-2 inhibitors to increase the susceptibility of latently HIV infected cells to cell death upon HIV reactivation and/or to kill HIV reactivating cells.

An ART can include any appropriate anti-retroviral agent or combination of anti-retroviral agents. Examples of anti-retroviral agents that can be used for ART include, without limitation, HIV integrase inhibitors, HIV protease inhibitors, and reverse transcriptase inhibitors. Examples of HIV integrase inhibitors include, without limitation, raltegravir (also known as Isentress or MK-0518), dolutegravir, and elvitegravir. Examples of HIV protease inhibitors include, without limitation, lopinavir and atazanavir. Examples of reverse transcriptase inhibitors include, without limitation, emtricitabine, rilpivirine, and tenofovir. In some cases, combinations of anti-retroviral agents can be formulated into a single dosage form (e.g., a single pill or capsule) such as Complera® (emtricitabine, rilpivirine, and tenofovir), Atripla® (efavirenz, emtricitabine, and tenofovir DF), Stribild® (cobicistat, elvitegravir, emtricitabine, and tenofovir), and triumeq® (abacavir, dolutegravir, and lamivudine).

Any appropriate Bcl-2 inhibitor or combination of Bcl-2 inhibitors (e.g., a combination of two, three, four, five, or more different Bcl-2 inhibitors) can be used as described herein. Examples of Bcl-2 inhibitors that can be used as described herein include, without limitation, ABT-199, ABT263, and Sabutoclax.

In some cases, one or more agents that facilitate cell death by a Casp8p41 induced cell death pathway can be used in place of or in combination with one or more Bcl-2 inhibitors to treat HIV infections as described herein. For example, one or more agents that facilitate cell death by a Casp8p41 induced cell death pathway (e.g., SM164) can be used alone to treat HIV infections. Agents that facilitate cell death by a Casp8p41 induced cell death pathway include those that can increase caspase 8 polypeptide levels or can facilitate post mitochondrial death signaling. Examples of agents that can increase caspase 8 polypeptide levels include, without limitation, those agents that can increase caspase 8 polypeptide levels in CD4 T cells such as IL-2. Examples of agents that can facilitate post mitochondrial death signaling include, without limitation, SMAC mimetics such as LBW242, SM164, and Birinapant.

In some cases, one or more Bcl-2 inhibitors can be formulated into a pharmaceutically acceptable composition for administration to a human having an HIV infection. For example, a therapeutically effective amount of ABT-199 can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules. A pharmaceutical composition containing one or more Bcl-2 inhibitors can be designed for oral or parenteral (including subcutaneous, intramuscular, intravenous, and intradermal) administration. When being administered orally, a pharmaceutical composition containing one or more Bcl-2 inhibitors can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

In some cases, a pharmaceutically acceptable composition including one or more Bcl-2 inhibitors can be administered systemically. For example, a composition containing a Bcl-2 inhibitor can be administered systemically orally or by injection to a human.

Effective doses can vary depending the route of administration, the age and general health condition of the human, excipient usage, the possibility of co-usage with other therapeutic treatments such as use of anti-retroviral agents and/or latency reversing agents, and the judgment of the treating physician.

An effective amount of a composition containing one or more Bcl-2 inhibitors can be any amount that increases the susceptibility of latently HIV infected cells to cell death upon HIV reactivation, thereby causing the latently HIV infected cells to die, without producing significant toxicity to the human. If a particular human fails to respond to a particular amount, then the amount of Bcl-2 inhibitor can be increased by, for example, two fold. After receiving this higher amount, the human can be monitored for both responsiveness to the treatment and toxicity symptoms, and adjustments made accordingly. The effective amount can remain constant or can be adjusted as a sliding scale or variable dose depending on the human's response to treatment. Various factors can influence the actual effective amount used for a particular application. For example, the frequency of administration, duration of treatment, use of multiple treatment agents, route of administration, and severity of the HIV infection may require an increase or decrease in the actual effective amount administered.

The frequency of administration of a composition containing one or more Bcl-2 inhibitors can be any frequency that increases the susceptibility of latently HIV infected cells to cell death upon HIV reactivation, thereby causing the latently HIV infected cells to die, without producing significant toxicity to the human. For example, the frequency of administration can be from about daily to about once a week. The frequency of administration can remain constant or can be variable during the duration of treatment. As with the effective amount, various factors can influence the actual frequency of administration used for a particular application. For example, the effective amount, duration of treatment, use of multiple treatment agents, route of administration, and severity of the HIV infection may require an increase or decrease in administration frequency.

An effective duration for administering a composition containing one or more Bcl-2 inhibitors can be any duration that increases the susceptibility of latently HIV infected cells to cell death upon HIV reactivation, thereby causing the latently HIV infected cells to die, without producing significant toxicity to the human. Thus, the effective duration can vary from several months to several years. In general, the effective duration for the treatment of an HIV infection as described herein can range in duration from about two months to about five years. Multiple factors can influence the actual effective duration used for a particular treatment. For example, an effective duration can vary with the frequency of administration, effective amount, use of multiple treatment agents, route of administration, and severity of the HIV infection being treated.

In some cases, a human having an HIV infection can be treated with one or more Bcl-2 inhibitors as described herein in combination with (a) one or more anti-retroviral agents, (b) one or more latency reversing agents, (c) one or more immunotherapeutic agents, (d) one or more vaccines (e.g., a vaccine formulated for assisting in the treatment of an HIV infection), (e) one or more nucleic acid-based therapies, (f) one or more polypeptides designed to restrict HIV expression such as TRIM5 alpha chimeric polypeptides, and (g) one or more advanced (e.g., third or later generation) chimeric antigen receptors expressed on CD8 T cells or NK cells designed to generate anti-HIV immunity. Examples of latency reversing agents that can be used in combination with one or more Bcl-2 inhibitors as described herein include, without limitation, HDAC inhibitors, phorbol esters, IL-2, bromodomain inhibitors, and those described elsewhere (Bullen et al., Nature Medicine, 20:425-429 (2014)). Examples of HDAC inhibitors that can be used as latency reversing agents include, without limitation, vorinostat, panabinostat, and valproic acid. Examples of phorbol esters that can be used as latency reversing agents include, without limitation, prostratin and PMA. An example of a bromodomain inhibitor that can be used as a latency reversing agent includes, without limitation, JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate).

Examples of immunotherapeutic agents that can be used in combination with one or more Bcl-2 inhibitors as described herein include, without limitation, IL-15, CD4 immunotoxin, and neutralizing anti-HIV antibodies. For example, a human having an HIV infection can be administered one or more Bcl-2 inhibitors as described herein and IL-15.

Examples of vaccines that can be used in combination with one or more Bcl-2 inhibitors as described herein include, without limitation, HIV tat or env antigens delivered by any number of platforms including genetic immunization, viral or virus like particle delivery, or delivery as recombinant proteins. The HIV antigens can be delivered with adjuvants such as CPG or GM-CSF. In some cases, a human having an HIV infection can be administered one or more Bcl-2 inhibitors as described herein and a HIV tat or env vaccine.

Examples of nucleic acid-based therapies that can be used in combination with one or more Bcl-2 inhibitors as described herein include, without limitation, nucleic acid molecules having the ability to reduce CCR5 polypeptide expression (e.g., siRNA molecules designed to reduce CCR5 polypeptide expression) and TALEN or CRISPR/Cas constructs designed to excise HIV DNA. For example, a human having an HIV infection can be administered one or more Bcl-2 inhibitors as described herein and an siRNA molecule designed to reduce CCr5 polypeptide expression.

In some cases, a human having an HIV infection can be treated with one or more Bcl-2 inhibitors as described herein in combination with one or more anti-retroviral agents plus any one or more of (a) one or more latency reversing agents, (b) one or more immunotherapeutic agents, (c) one or more vaccines (e.g., a vaccine formulated for assisting in the treatment of an HIV infection), and (d) one or more nucleic acid-based therapies.

In some cases, the level of HIV infected cells within a human being treated can be monitored during the course of treatment. Any appropriate method can be used to determine the level of HIV infected cells within a human. For example, the level of HIV infected cells within a human can be assessed using PCR based detection methods (nested or un-nested) for detecting HIV DNA, quantitative viral outgrowth assays (QVOA) for measuring replication competent HIV levels, or TILDA (Tat/rev; Induced Limiting Dilution Assay) that can measure the frequency of cells with multiply spliced HIV RNA as a surrogate for replication competent HIV.

The invention will be further described in the following examples, which do not limit the scope of the invention described in the claims.

### EXAMPLES

### Example 1—Bcl-2 Inhibition Facilitates Killing of HIV Infected Cells and Reduces Cell Associated HIV DNA

### Cell Culture

Jurkat cells and HEK 293T cells were obtained from American Type Culture Collection (Manassas, VA). Jurkat/Bcl-2 cells were created by transfecting Jurkat with a pcDNA-3 encoding Bcl-2 (obtained from Dr. Stan Korsmeyer), selecting for 30 days, and assessing Bcl-2 expression by immunoblotting. HIV-uninfected PBMCs were harvested by ficoll gradient centrifugation from Leukocyte Reduction System apheresis chambers provided by healthy volunteer blood donors. Primary bulk CD4 T cells were isolated using RosetteSep™ Human CD4⁺ T Cell Enrichment Cocktail (StemCell Technologies), activated for 24 hours with 1 μg/mL phytohemagglutinin, washed in medium, and incubated for 48 hours with 50 units/mL IL-2 prior to HIV infection. TCM and TEM were treated with CH-11 anti-Fas antibody (1 μg/mL), cycloheximide (CHX, 30 μM), etoposide (20 μM), camptothecin (20 μM), carbonyl cyanide 3-chlorophenyl-hydrazone (CCCP, 1 μM), or H₂O₂ (0.35 mM) overnight to induce cell death.

### Plasmid and Peptide Preparation

Casp8p41 in pEGFP, pcDNA3, and pGEX-4T were described elsewhere (Nie et al., Open Virol. J., 2:1-7 (2008); and Bren et al., PLoS One, 3:e2112 (2008)). Plasmids encoding Bcl-2, Bcl-xL, and Mcl-1 were described elsewhere (Smith et al., J Biol. Chem., 286:17682-17692 (2011)). The indicated Casp8p41 and BCL-2 mutations were introduced using site-directed mutagenesis (Agilent Technologies, Santa Clara, CA) and confirmed by sequencing. The Casp8p41 BH3-like peptide (DMNLLDIFIEMEKRVILGEGKLDILKRVCAQ; SEQ ID NO:1), N-terminal control peptide (MDFSRNLYDIGEQLDSEDLASLK; SEQ ID NO:2) and Bim BH3 peptide (Dai et al., J. Cell. Biol., 194:39-48 (2011)) were synthesized by solid phase synthesis.

### Transfection

HEK 293T cells were transfected using Lipofectamine (Invitrogen, Carlsbad, CA) according to manufacturer's protocol. Jurkat cells were transfected using a square wave electroporator (BXT, San Diego, CA) at 320 V.

### Immunoprecipitation and Immunoblotting

293T cells transfected with empty vector, HA-Casp8p41, or HA-Casp8p41EK were collected after 24 hours, washed with PBS, and lysed (20 mM Tris/HCl pH 7.5, 150 mM NaCl, 1% CHAPS, 2 μg/mL aprotinin, 10 μg/mL leupeptin, 2 μg/mL pepstatin, and 1 mM PMSF) for 10 minutes on ice and centrifuged at 15000 g for 5 minutes at 4° C. Aliquots containing 500 μg protein were precleared with 25 μL Protein A/G-agarose (Santa Cruz Biotechnology, Santa Cruz, CA), incubated with 5 μg anti-Bcl-2 clone C22 (Santa Cruz Biotechnology, Santa Cruz, CA) overnight at 4° C., supplemented with 10 μL Protein-A/G agarose. Beads were washed with lysis buffer 3 times in 10 fold the volume of the beads. Bound protein was eluted and subjected to SDS-PAGE followed by immunoblotting as described elsewhere (Sainski et al., J. Virol., 85:7965-7975 (2011)). Primary antibodies used were: anti-HA peroxidase high affinity 3F10 (Roche, St. Louis, MO) or anti-Bcl-2 clone C22, Mcl-1 clone 22, or Bcl-xL clone s18 (Santa Cruz Biotechnology, Santa Cruz, CA).

### Protein Expression and Purification

After plasmids encoding GST tagged proteins were transformed into E. coli BL21 or DH5α by heat shock, bacteria were grown to an optical density of 0.8 and induced with 1 mM IPTG for 3 hours at 37° C. Bacteria were disrupted by freezing and thawing in calcium- and magnesium-free Dulbecco's phosphate buffered saline (PBS) containing 0.1% Triton X-100, 2 μg/mL aprotinin, 10 μg/mL leupeptin, 2 μg/mL pepstatin, and 1 mM PMSF, then sonicated three times for 15 sec/min on ice. GST-tagged proteins were purified with glutathione-agarose (Thermo Fisher Scientific, Rockford, IL).

### Surface Plasmon Resonance (SPR)

Proteins for SPR were further purified by FPLC on Superdex S200, concentrated in a centrifugal concentrator (Centricon, Millipore), dialyzed against Biacore buffer (10 mM HEPES (pH 7.4), 150 mM NaCl, 0.05 mM EDTA, and 0.005% (w/v) Polysorbate 20) and stored at 4° C. for <48 hours before use. Binding assays were performed at 25° C. on a Biacore 3000 biosensor after proteins were immobilized on a CM5 chip (GE Healthcare). Ligands were injected at 30 μL/minute for 1 minute in Biacore buffer. Bound protein was allowed to dissociate in Biacore buffer at 30 μL/minute for 10 minutes and then desorbed with 2 M MgCl2. Binding kinetics were derived using BIA evaluation software (Biacore, Uppsala, Sweden).

### Flow Cytometry

Immunophenotyping of T cell subsets was performed using multicolor flow cytometry with monoclonal antibodies to human CD3 (Alexa700, BD Pharmingen), CD4 (FITC, BD Pharmingen), CD8 (Pacific Blue, BD Pharmingen), CD27 (PE, BD Pharmingen), and CD45RO (ECD, Beckman Coulter). Central memory T cells (T_{CM}) were defined as CD3⁺CD4⁺CD27⁺CD45RO⁺, and effector memory T cells (T_{EM}) were defined as CD3⁺CD4⁺CD27⁻CD45RO^{+/-} (Brenchley et al., J. Exp. Med., 200:749-759 (2004)). Intracellular expression of Casp8p41 was assessed as described elsewhere (Cummins et al., AIDS Res. Hum. Retroviruses, 30:476-479 (2014)). Cell death was measured using LIVE/DEAD® Fixable Aqua dead cell stain (Invitrogen) or TUNEL (Roche) according to manufacturers' protocols. Cell proliferation was measured using a CellTrace™ CFSE Cell Proliferation Kit (Life Technologies) according to manufacturer's protocol. FACS analysis was performed on either a FACScan or LSRII flow cytometer (BD Biosciences) based on multiparameter needs. FACS data were analyzed using FlowJo software (Tree Star Inc).

### HIV Infections

Jurkat and Jurkat/Bcl-2 cells were infected overnight, while primary PHA-activated CD4 T cells were infected for 6 hours with HIV-1IIIb (NIH AIDS Reagent Program). Aliquots of the same infectious supernatant were used for all experiments. Cells were then washed three times and incubated in fresh medium. At the indicated time points, HIV-1 p24 in the cell culture supernatant was measured by RETROTEK™ ELISA kits (Zeptometrix Corporation) according to manufacturer's protocol. Cell associated HIV-1 DNA was assayed using a modification of the protocol described elsewhere (Liszewski et al., Methods, 47:254-260 (2009)). Briefly, total DNA was extracted using the Qiagen DNeasy Blood and Tissue kit (Hilden, Germany) and analyzed by a real-time polymerase chain reaction (PCR) assay specific for HIV-LTR and β-globin. A standard curve of pNL4-3 plasmid from 10⁶ through to 10 copies was used as an internal control. 300 nM of the sense primer RU5-F 5′-TTAAGCCTCAATAAAGCTTGCC-3′ (SEQ ID NO:3) and antisense primer RU5-R 5′-GTTCGGGCGCCACTGCTAGA-3′ (SEQ ID NO:4) were used in conjunction with 300 nM dual-labeled fluorogenic TaqMan probe 5′-FAM-CCAGAGTCACACAACAGACGG GCACA-TAMRA-3′ (SEQ ID NO:5). For a 20 μL reaction, 10 μL of gene expression master mix (Applied Biosystems, Carlsbad, CA) was used with 5 μL of genomic DNA. PCR conditions involved one cycle of 95° C. for 3 minutes followed by 45 cycles of 95° C. for 15 seconds and 60° C. for 1 minute. Total HIV-1 DNA was compared and normalized with genomic DNA, determined by β-globin. HIV-1 proviral DNA levels were expressed as HIV-1 copies/β-globin genomic equivalent of 10⁶ cells.

### Next Generation Sequencing

Primary T_{CM} and T_{EM} were isolated using CD4⁺ Central Memory T Cell Isolation and CD4⁺ Effector Memory T Cell Isolation Kits (MACS Miltenyi Biotec). After RNA was extracted (Qiagen RNA easy kit), the quality of total RNA samples was assessed by Agilent Bioanalyzer (Santa Clara, CA). RNA libraries were prepared according to the manufacturer's instructions for the TruSeq RNA Sample Prep Kit v2 (Illumina, San Diego, CA). Poly-A mRNA was purified from total RNA using oligo dT magnetic beads. The purified mRNA was fragmented at 95° C. for 8 minutes, eluted from the beads, and primed for first strand cDNA synthesis. The RNA fragments were then copied into first strand cDNA using SuperScript III reverse transcriptase and random primers (Invitrogen, Carlsbad, CA). Second strand cDNA synthesis was performed using DNA polymerase I and RNase H. The double-stranded cDNA was purified using a single AMPure XP bead (Agencourt, Danvers, MA) clean-up step. cDNA ends were repaired and phosphorylated using Klenow, T4 polymerase, and T4 polynucleotide kinase followed by a single AMPure XP bead clean-up. The blunt-ended cDNAs were modified to include a single 3′ adenylate (A) residue using Klenow exo-(3′ to 5′ exo minus). Paired-end DNA adaptors (Illumina) with a single “T” base overhang at the 3′ end were ligated to the ‘A tailed’ cDNA population. Unique indexes, included in the standard TruSeq Kits (12-Set A and 12-Set B) were incorporated at the adaptor ligation step for multiplex sample loading on the flow cells. The resulting constructs were purified by two consecutive AMPure XP bead clean-up steps. The adapter-modified DNA fragments were then enriched by 12 cycles of PCR using primers included in the Illumina Sample Prep Kit. Libraries were loaded onto paired end flow cells at concentrations of 8-10 pM to generate cluster densities of 700,000/mm² following Illumina's standard protocol using the Illumina cBot and cBot Paired end cluster kit version 3. The flow cells were sequenced as 51×2 paired end reads on an Illumina HiSeq 2000 using TruSeq SBS sequencing kit version 3 and HCS v2.0.12 data collection software. Base-calling was performed using Illumina's RTA version 1.17.21.3. Data were analyzed according to a Mayo Clinic developed protocol for analyzing RNA-Sequencing data (see, e.g., the World Wide Web at “biomedcentral.com/content/pdf/1471-2105-15-224.pdf”).

Once the gene counts were provided by MAP-RSeq, a differential expression and gene set enrichment analysis was performed. DESeq33, an r package, was used to normalize and quantify log fold change, p-values, and the false discovery rate between the groups. A hypergeometric test was then performed on all targets with an absolute log 2 fold change of greater than 1 to investigate enrichment of genes associated with cell death and cell proliferation. Cell death and cell proliferation genes were gathered from DeathBase (Diez et al., Cell Death Differ., 17:735-736 (2010), Gene Ontology (Ashburner et al., Nat. Genet., 25:25-29 (2000), and KEGG pathways (Kanehisa and Goto, Nucleic Acids Res., 28:27-30 (2000)).

### Statistical Analysis

Mean values of experimental results were compared by t-tests (non-paired for transformed cell line experiments, and paired for primary cell experiments) or ANOVA or Friedman tests as appropriate. Results of time course experiments were compared by area under the curve analyses, with mean AUC values compared by t-tests. P<0.05 considered statistically significant.

### Molecular Modeling

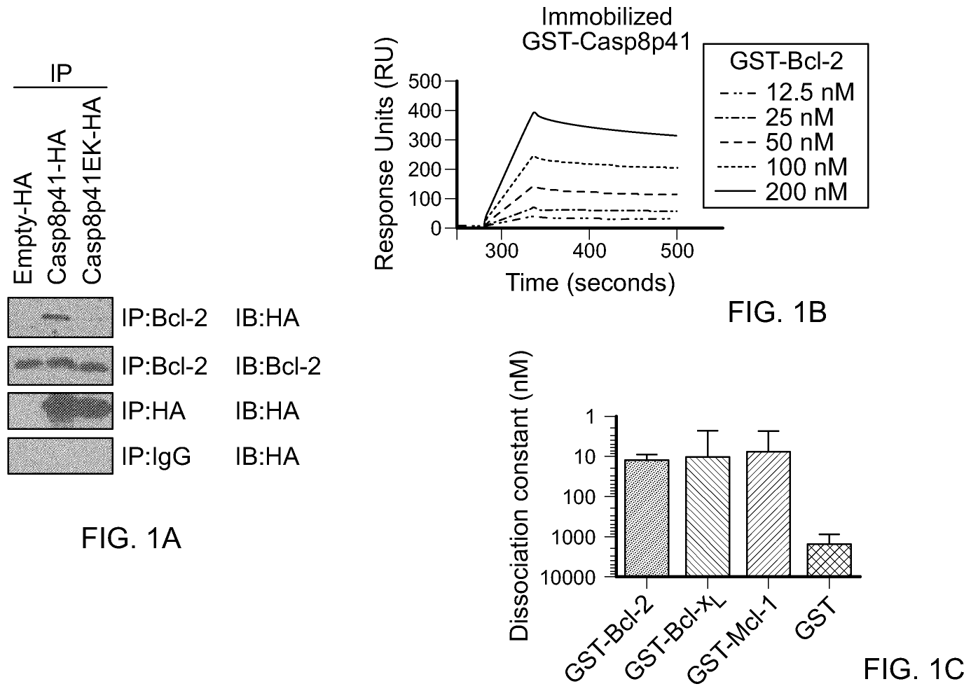

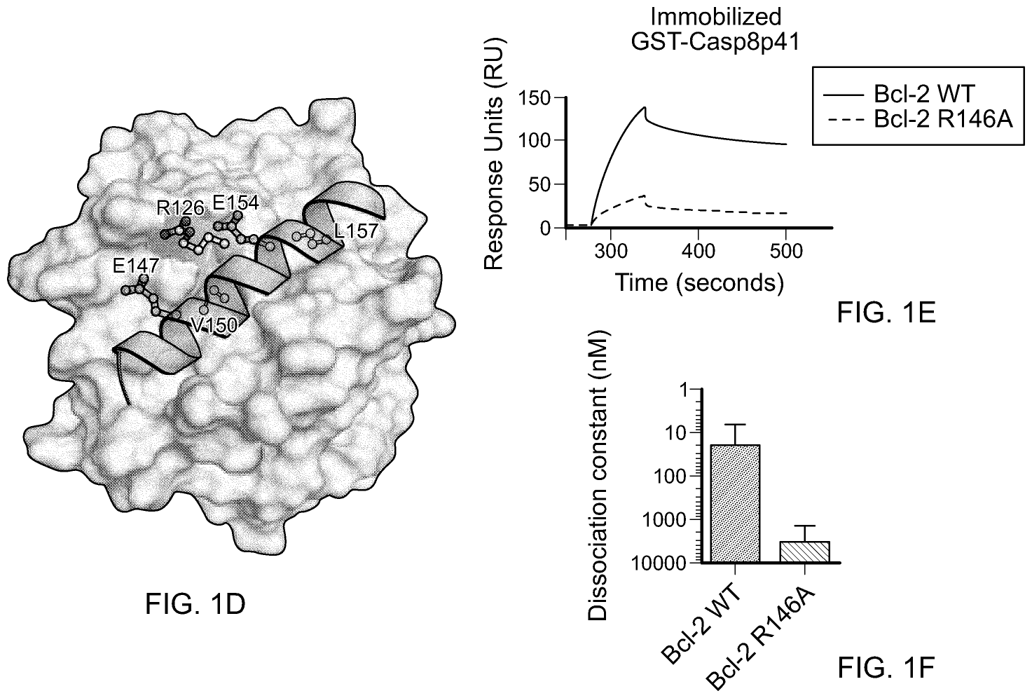

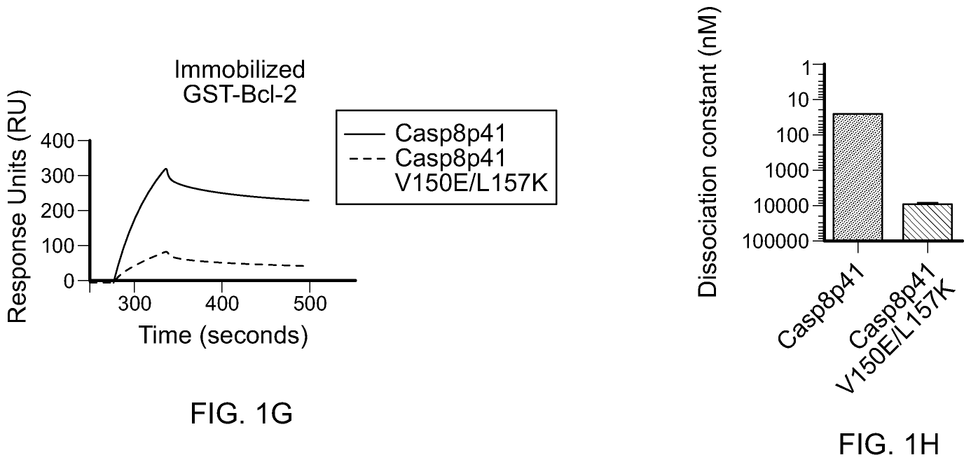

The initial structure of the Casp8p41·Bcl-2 complex was generated by manually docking the activator domain of Casp8p41 (residues 142-162) in the α-helical conformation into the vacated BH3-binding groove of Bcl-2 (residues 50-203) that was taken from the crystal structure of human Bcl-2 (Protein Data Bank ID: 4AQ3; residue 9 of the 4AQ3 structure corresponds to residue 50 of the human Bcl-2 sequence of NCBI Accession ID of P10415). This manual docking placed V150^{Casp8p41} in the proximity of L137^{Bcl-2} and L157^{Casp8p41} close to V148^{Bcl-2}. All Glu, Asp, Arg, and Lys residues were treated as GLU, ASP, ARG, and LYS, respectively. His94^{Bcl-2} was treated as HIE, and all other His residues were treated as HIP. The topology and coordinate files of the complex were generated by using LEAP of AmberTools 1.5 (University of California, San Francisco). The complex was refined by energy minimization using SANDER of AMBER 11 (University of California, San Francisco) with a dielectric constant of 1.0 and 200 cycles of steepest-descent minimization followed by 300 cycles of conjugate-gradient minimization using AMBER forcefield FF12MC. Developed by Yuan-Ping Pang, FF12MC is based on AMBER forcefield FF99 with changes of (i) reducing all atomic masses by tenfold to improve configurational sampling (Pang, Biochem. Biophys. Res. Commun., 452: 588-592 (2014)), (ii) shortening C—H bonds by 10-14% (1.09 Å to 0.98 Å for the aliphatic; 1.08 Å to 0.93 Å for the aromatic) (Pang, Biochem. Biophys. Res. Commun., 458:352-355 (2015)), and (iii) zeroing torsion potentials involving a nonperipheral sp3 atom with reduction of the 1-4 interaction scaling factors of protein backbone torsions φ and ψ (from 2.00 to 1.00 for the van der Waals interaction; from 1.20 to 1.18 for the electrostatic interaction) (Pang, Biochem. Biophys. Res. Commun., 457: 183-186 (2015)). The energy minimized complex was then solvated by using the LEAP module with 5946 TIP3P water molecules with the distance parameter of 8.2 Å for the solvatebox command. The solvated complex system was energy-minimized for 100 cycles of steepest-descent minimization followed by 900 cycles of conjugate-gradient minimization to remove close van der Waals contacts in the system, then heated from 0 to 300 K at a rate of 10 K/ps under constant temperature and constant volume, and finally simulated in ten unique, independent 10-ns molecular dynamics simulations using PMEMD of AMBER 11 with FF12MC. The ten unique seed numbers for initial velocities of Simulations 1-10 are 1804289383, 846930886, 1681692777, 1714636915, 1957747793, 424238335, 719885386, 1649760492, 596516649, and 1189641421, respectively. These all-atom, isothermal-isobaric MD simulations used (i) a dielectric constant of 1.0, (ii) the Berendsen coupling algorithm (Berendsen et al., J. Chem. Phys., 81:3684-3690 (1984)), (iii) the Particle Mesh Ewald method to calculate long-range electrostatic interactions (Darden et al., J. Chem. Phys., 98:10089-10092 (1993), (iv) a time step of 1.0 fs, (v) SHAKE-bond-length constraints applied to all the bonds involving the H atom, (vi) a protocol to save the image closest to the middle of the “primary box” to the restart and trajectory files, (vii) a formatted restart file, (viii) a nonbonded cutoff of 8.0 Å, and (ix) default values of all other inputs of PMEMD. All simulations were performed on ten 12-core Apple Mac Pro microcomputers with Intel Westmere (2.40 GHz) processors. A cluster analysis of all conformations saved at 100-ps intervals from the ten 10-ns MD simulations was performed using PTRAJ of AmberTools 1.5 with the average-linkage algorithm (Shao et al., J Chem. Theory Comput., 3:2312-2334 (2007)) (epsilon of 2.0 Å and root mean square coordinate deviation on all Cu atoms of the BH3-like domain). The occurrences of the three most populated clusters are 67%, 6%, and 5%, respectively. The structure displayed in FIG. 1D is the representative conformation of the most populated cluster identified by PTRAJ.

### Results

During acute HIV infection, CD4 T cell death is initiated when viral RNA is detected by RIG-I (Solis et al., J. Virol., 85:1224-1236 (2011); and Jiang et al., Nature 479, 423-427 (2011), reverse transcribed viral DNA is detected by IFI-16 (Doitsh et al., Nature, 505:509-514 (2014); and Monroe et al., Science, 343:428-432 (2014)), or HIV integrase-induced nicking of the host DNA is detected by DNA-PK (Cooper et al., Nature, 498:376-379 (2013)). When these death mechanisms fail, HIV integrates into the host genome and may enter a latent state in which the virus is transcriptionally silent, replication competent, and resistant to current therapies or immune attack. Upon viral reactivation from latency, which occurs during T cell activation, these innate immune sensing mechanisms are not activated, but HIV protease-mediated cleavage of the host protein procaspase 8 (Nie et al., Cell Death Differ., 9:1172-1184 (2002); and Nie et al., Open Virol. J., 2:1-7 (2008)) generates a fragment (Casp8p41) with an α-helical domain that directly activates the pro-apoptotic mitochondrial protein Bak to facilitate HIV-induced killing (Sainski et al., J Cell. Biol., 207:159 (2014)).

As described herein, central memory CD4 T cells, the principal reservoir of HIV, are intrinsically resistant to cell death due, in part, to elevated expression of Bcl-2. Further, it was demonstrate that Bcl-2 binds Casp8p41, inhibits Casp8p41-mediated killing, and diminishes T cell death following acute HIV infection in vitro, resulting in increased HIV production. Conversely, antagonizing Bcl-2 selectively promoted death of Casp8p41-containing cells and diminished HIV DNA following either acute HIV infection or HIV reactivation from latency ex vivo. These results demonstrate that apoptosis sensitization followed by HIV reactivation can reduce HIV burden.

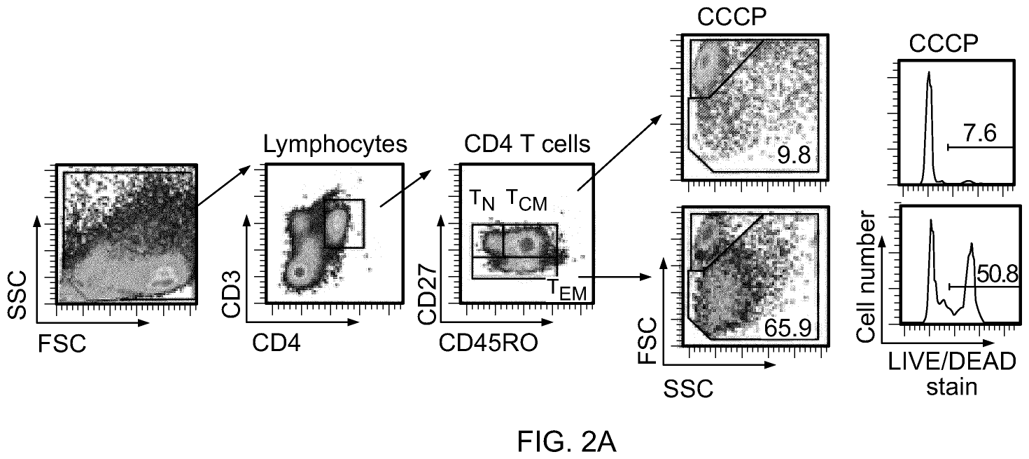

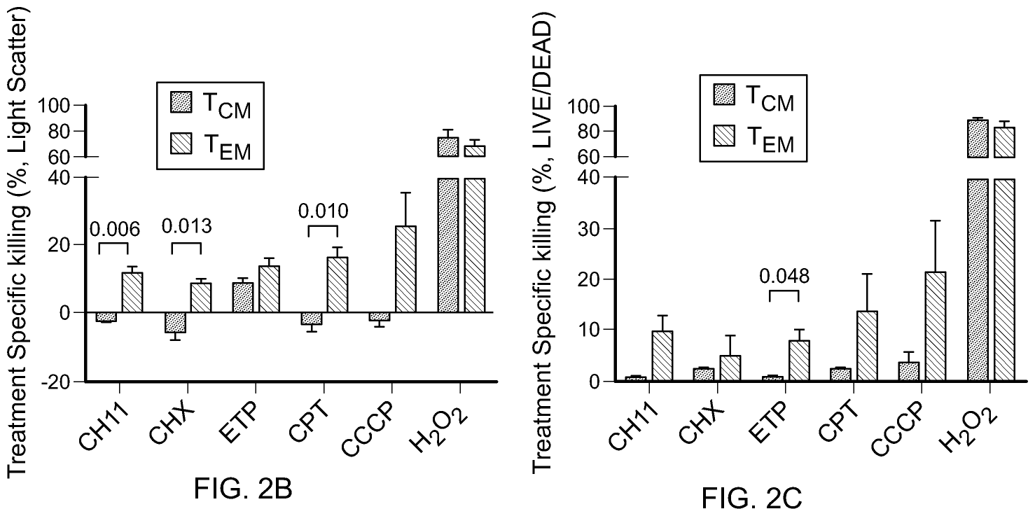

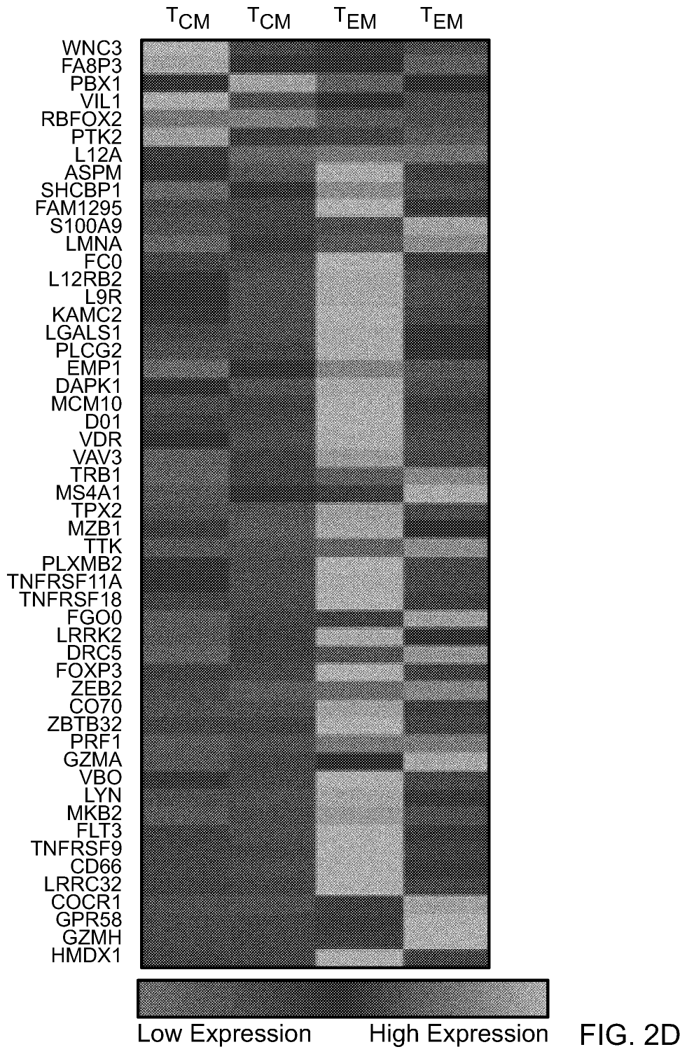

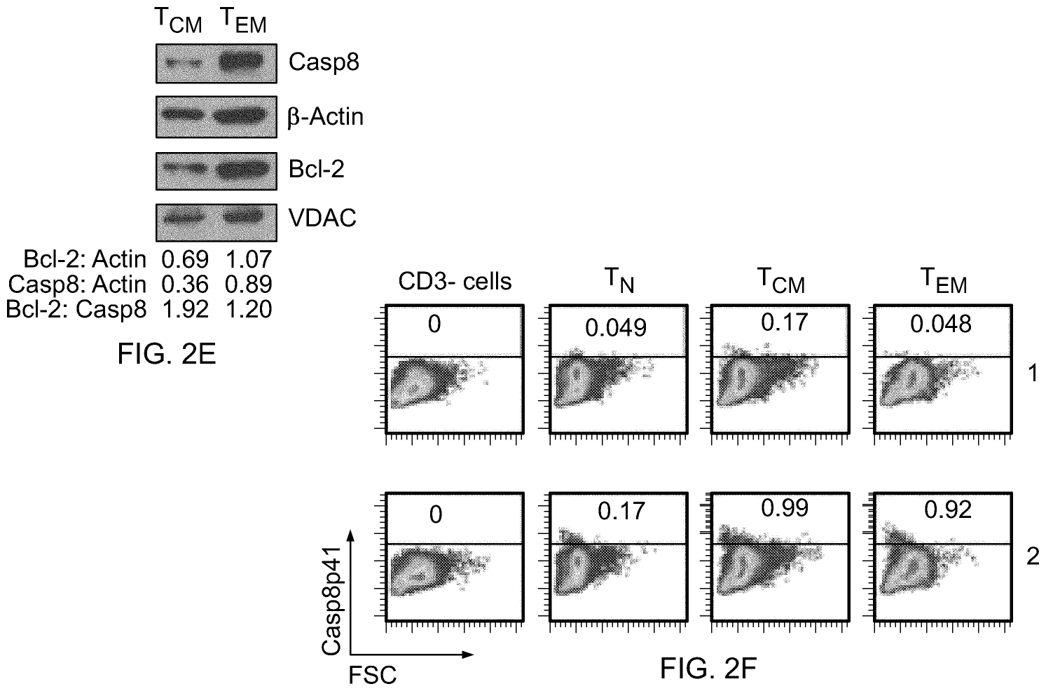

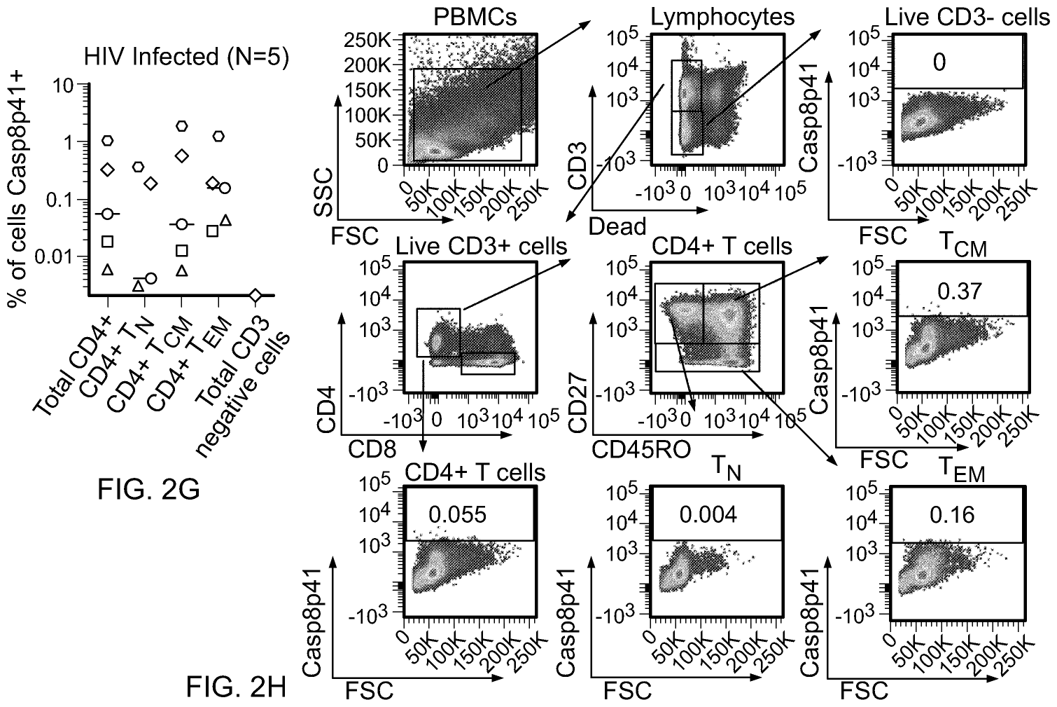

Once integrated into host DNA, HIV can remain in a latent state for years. In patients on suppressive ART, T_{CM}, which are long lived and proliferate in response to antigenic re-stimulation, constitute the principal HIV reservoir (Jaafoura et al., Nat. Commun., 5:5407 (2014)). By contrast, T_{EM} are a lesser reservoir (Chomont et al., Nat. Med., 15:893-900 (2009)) and have a shorter half-life (Macallan et al., J Exp. Med., 200:255-260 (2004)). To assess whether the longer life span of T_{CM} reflects intrinsic resistance to death stimuli, PBMCs from uninfected donors were treated with proapoptotic stimuli. T_{CM} were less susceptible than T_{EM} to Fas ligation and triggers of the mitochondrial apoptotic pathway (FIG. 2A-2C). RNAseq performed on T_{CM} and T_{EM} revealed increased expression of six proliferation genes and decreased expression of 46 cell death genes by at least 2-fold in T_{CM} (p=6.5×10⁻⁶ for enrichment of the death and proliferation gene sets, FIG. 2D), providing a potential explanation for the apoptosis resistance of T_{CM}. Importantly, eradication of HIV required elimination of these apoptosis resistant cells if they harbor the virus.

The results were consistent with other gene expression data (GSE61697, Gene Expression Omnibus) showing higher expression of BCL-2 and lower expression of four other genes that impact T cell survival (TNFRSF9, TNFRSF11 A, PRF1 and GZMA) in T_{CM} compared to T_{EM} (FIG. 2I; Takeshita et al., Clin. Immunol., 159:107-117 (2015)).

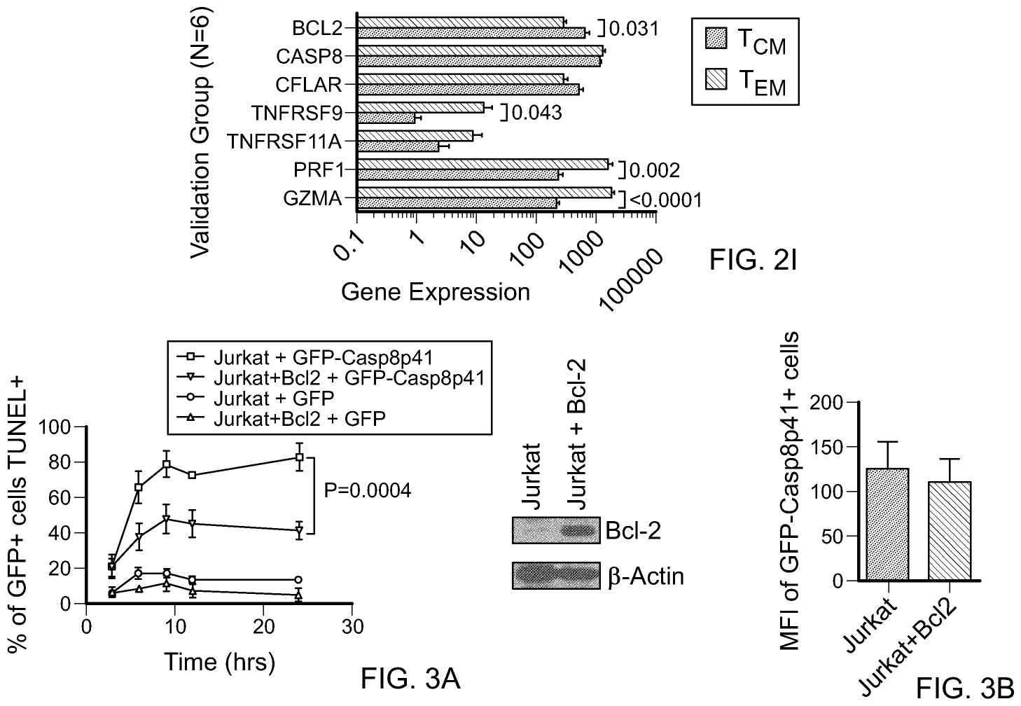

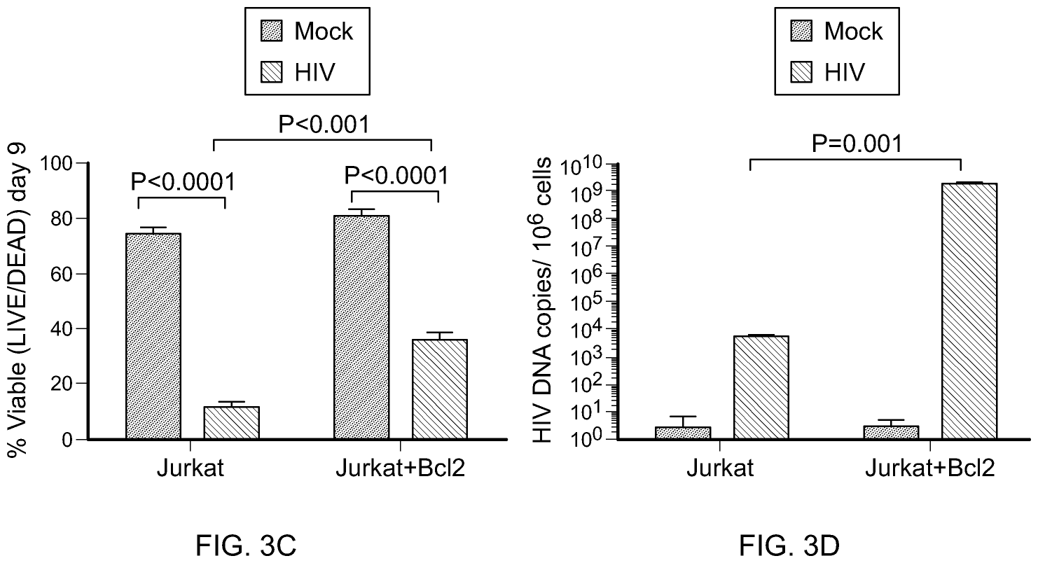

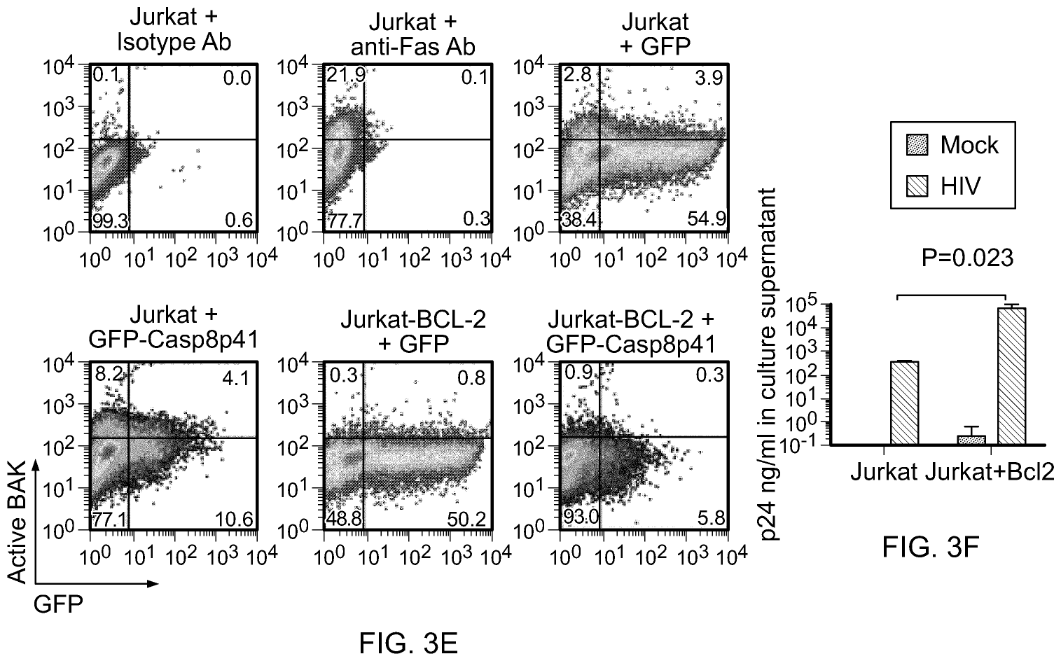

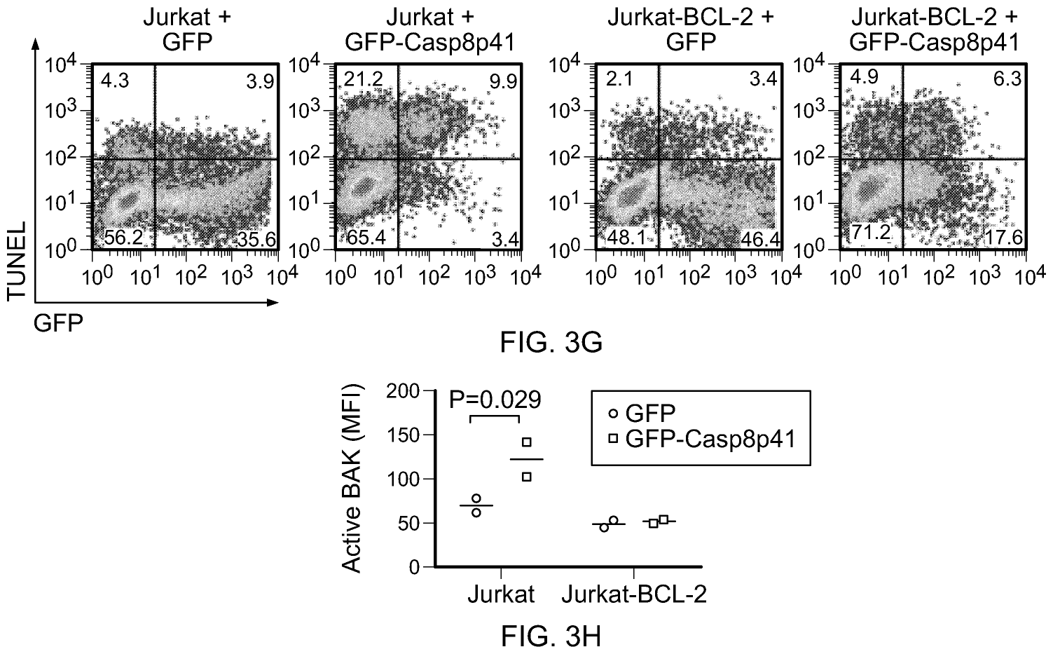

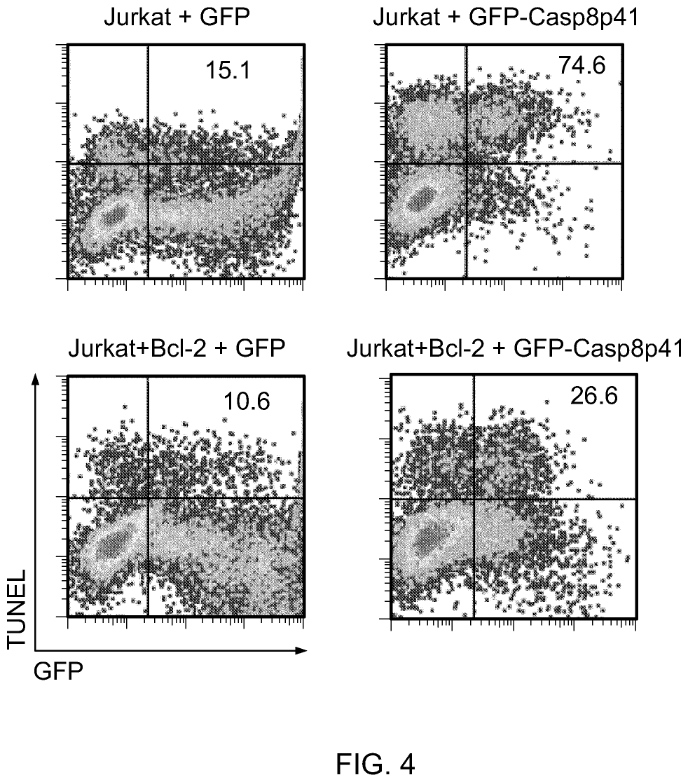

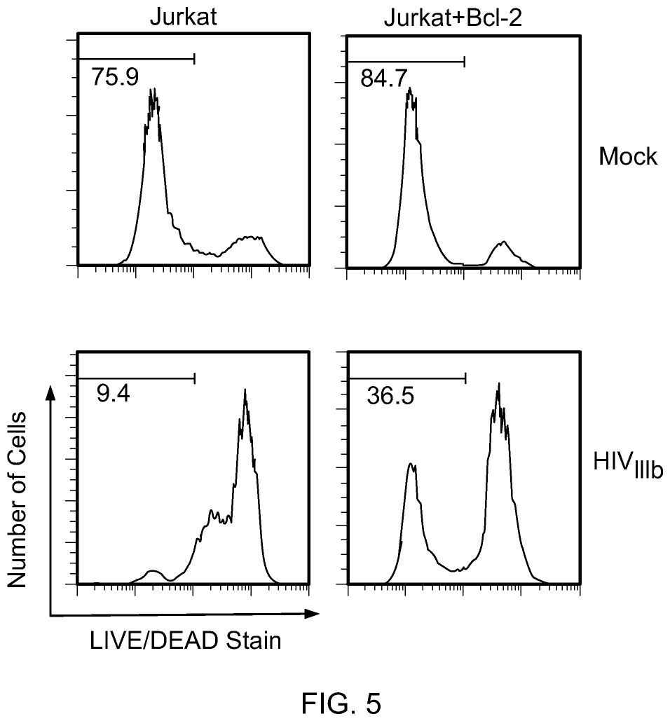

In light of results showing that Casp8p41 can act like a BH3-only protein, there are two possible reasons why T_{CM} that reactivate HIV might not die: Either Casp8p41 is not generated, or the proapoptotic effects of Casp8p41 are antagonized by intrinsic resistance mechanisms. Since T_{CM} express Caspase 8 (FIG. 2E) and flow cytometry readily detected Casp8p41 in T_{CM} from HIV-infected patients (FIG. 2F), whether there is a block to Casp8p41-mediated apoptosis was assessed. Because the Bcl-2:Caspase8 ratio is higher in T_{CM} than T_{EM} (FIG. 2E), the impact of Bcl-2 on Casp8p41-mediated killing was assessed in Jurkat T cells. After transfection with EGFP-Casp8p41, successfully transfected cells were identified by their EGFP fluorescence and assayed for cell killing using terminal deoxynucleotidyltransferase dUTP nick end labeling (TUNEL). Bcl-2 overexpression diminished the number of EGFP-Casp8p41⁺ cells that were TUNEL⁺ (FIG. 3A, p=0.004) despite similar Casp8p41 expression (FIGS. 3B and 4). If Bcl-2 overexpressing cells die less following HIV infection, then more cells should survive to produce more progeny HIV virions. Consistent with this prediction, Bcl-2 overexpression increased cell survival following HIV infection, cell associated HIV DNA, and HIV p24 production (FIGS. 3C-E and 5), demonstrating that altering Bcl-2 fundamentally impacts the magnitude of viral replication and number of HIV-infected cells.

In addition, since T_{CM} express detectable levels of Caspase 8 (FIG. 2E), T_{CM} from five chronically HIV-infected people were analyzed to determine whether they generate Casp8p41 in vivo (FIGS. 2G and 2H). Flow cytometry readily detected Casp8p41 in 0.006 to 1.86% of T_{CM}, as well as 0 to 0.36% of TN and 0.006 to 1.22% of T_{EM}; but was not detected in CD3 negative cells. This frequency of Casp8p41 positivity in resting memory CD4 T cells was similar to the frequency of Gag sequences in memory CD4 T cells found in another study that detected 100 to 10,000 Gag copies per 10⁵ highly purified sorted memory CD4 T cells from HIV infected patients (Brenchley et al., J. Virol., 78:1160-1168 (2004)).

### BCL-2 Inhibits Casp8p41-Induced Cell Death

To assess whether BCL-2 alters the ability of Casp8p41 to kill cells, the responses of parental Jurkat T cells versus Jurkat cells stably overexpressing BCL-2 (Jurkat-BCL-2) were compared to transient GFP-Casp8p41 expression. After transfection, Casp8p41-induced apoptosis, as measured by TUNEL positivity (FIGS. 3G and 3A), was greater in parental Jurkat cells than Jurkat-BCL-2 cells (Mean AUC 1500 vs 870 [95% CI of difference −790, −480; P=0.004]) despite similar Casp8p41 expression (FIG. 3B). Thus, cells overexpressing BCL-2 die less in response to Casp8p41, indicating that BCL-2 antagonizes Casp8p41 induced killing.

To determine whether BCL-2 overexpression impacts Casp8p41-mediated BAK activation, Jurkat or Jurkat-BCL-2 cells were transfected with GFP or GFP-Casp8p41 and stained with conformational specific antibodies that recognize activated BAK, but not inactive BAK. As expected, GFP-Casp8p41⁺ expressing parental Jurkat cells (identified as GFP⁺ by flow cytometry) contained more activated BAK than parental cells expressing GFP alone (MFI difference 52±16, P=0.029; FIGS. 3H and 3E). In contrast, GFP-Casp8p41⁺ Jurkat-BCL-2 cells did not exhibit increased BAK activation compared to GFP alone (MFI difference 3.5±16, P=0.834), indicating that BCL-2 prevents BAK activation in response to Casp8p41 expression. Accordingly, the overall number of activated BAK⁺ cells after GFP-Casp8p41 transfection was lower in Jurkat-BCL-2 cells compared to Jurkat cells (P=0.045). Thus, BCL-2 inhibited Casp8p41-induced BAK activation.

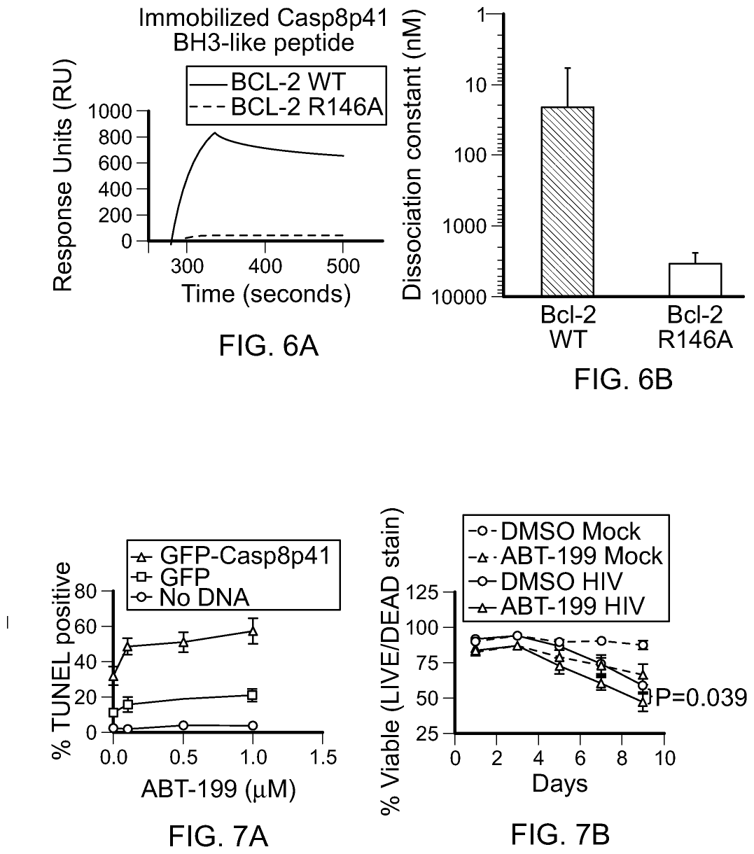

Because Casp8p41 contains an alpha helical activator domain that can bind the BH3 binding groove of Bak (Sainski et al., J Cell. Biol., 207:159 (2014)), whether the same domain can bind antiapoptotic Bcl-2 family members as well was evaluated. In initial experiments, Bcl-2 was immunoprecipitated from lysates of 293T cells transfected with empty vector, HA-Casp8p41, or HA-Casp8p41 Val150Glu/Leu157KLys (a variant with decreased affinity for Bak (Sainski et al., J Cell. Biol., 207:159 (2014))). An interaction of Bcl-2 with HA Casp8p41 that was reduced by the Val150Glu/Leu157KLys modification was observed (FIG. 1A). In further experiments, SPR demonstrated equilibrium mean dissociation constants (Kds) of 13 nM for Casp8p41 binding to purified recombinant Bcl-2, 11 nM for Bcl-xL, and 8 nM for Mcl-1 (FIG. 1B-C). Purified activator domain peptide also bound Bcl-2, providing evidence that the same Casp8p41 domain is responsible for binding both Bak and Bcl-2 (FIG. 6). Using multiple low-mass molecular dynamic simulations (Pang, Biochem. Biophys. Res. Commun., 452:588-592 (2014)), a three-dimensional model of that peptide bound in the BH3 binding groove of Bcl-2 (FIG. 1D) that predicts a critical interaction of Bcl-2 Arg146 with Casp8p41 Glu147 and Glu154 was determined. Substitution of Ala for Bcl-2 Arg146, which inhibits Bcl-2·Bim interaction (Smith et al., J Biol. Chem., 286:17682-17692 (2011)), decreased the affinity of Bcl-2 for the Casp8p41 activator peptide (FIG. 6) or full length Casp8p41 250-fold (FIG. 1E-F), confirming that Casp8p41 binds the Bcl-2 BH3 binding groove. The model also predicted that Casp8p41 Val150 and Leu157 bind in the two hydrophobic regions in the Bcl-2 BH3 binding groove. Consistent with this prediction, the affinity of Casp8p41 for Bcl-2 was reduced over 300-fold by the Val150Glu/Leu157Lys Casp8p41 modification (FIG. 1G-H). These results indicate that the Casp8p41 activator domain can interact with the BH3 binding groove of anti-apoptotic Bcl-2 family members as well as Bak.

Venetoclax is a Clinically Relevant BCL-2 Antagonist that Impacts Casp8p41-Mediated Killing

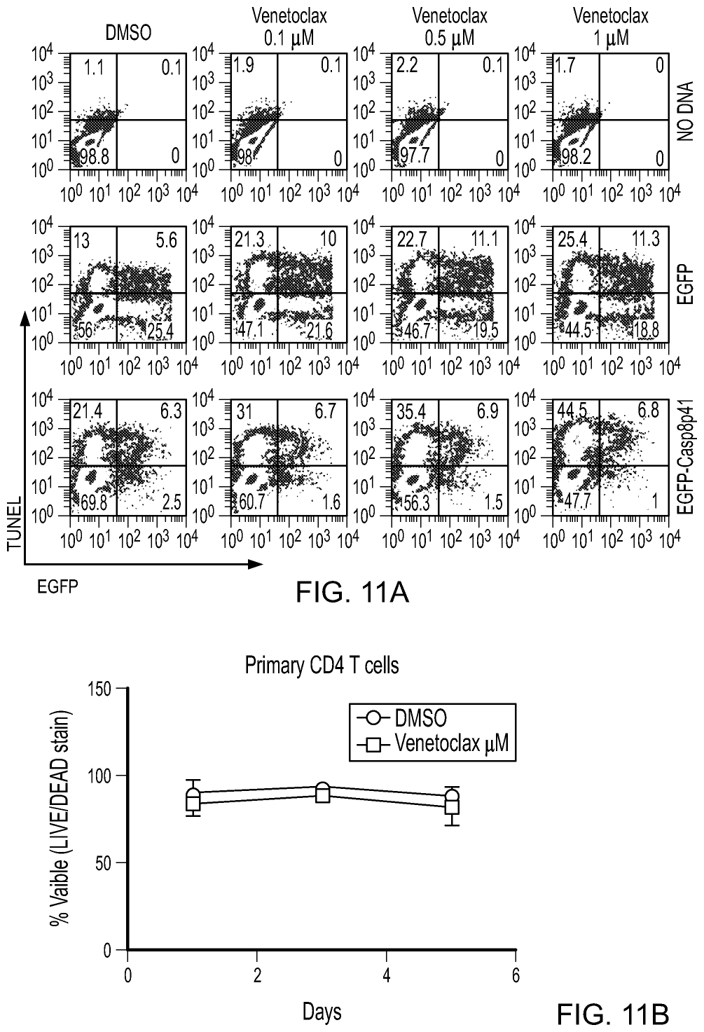

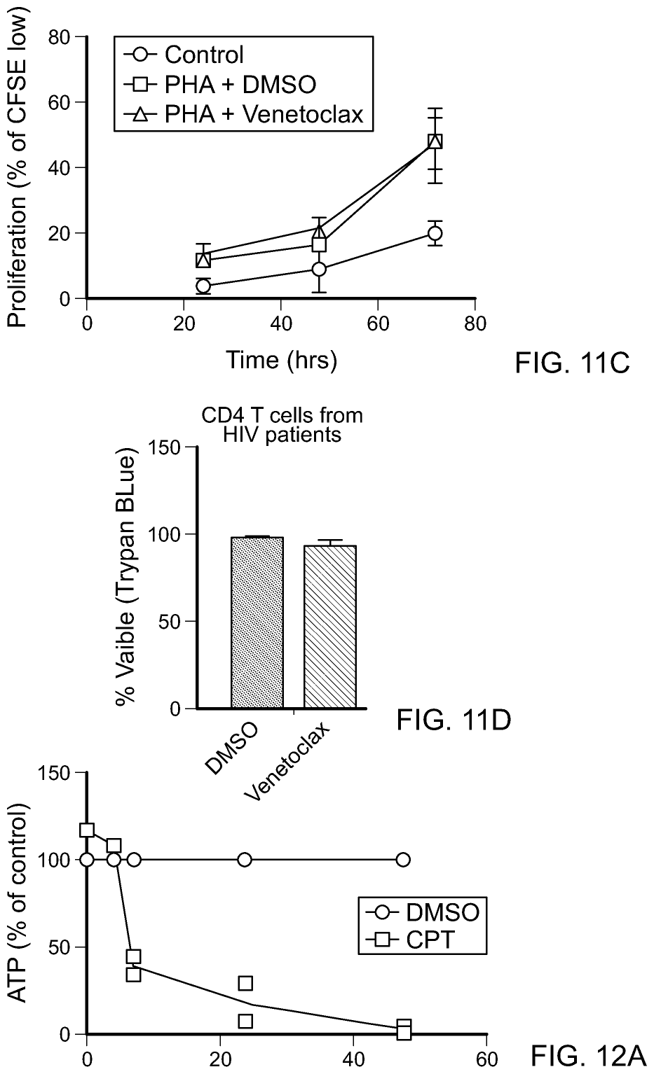

Venetoclax (formerly known as ABT-199), potently and selectively inhibits BCL-2 binding to BH3 domains (Souers et al., Nat. Med., 19:202-208 (2013)), is well tolerated in early phase clinical trials (Correia et al., Biochim Biophys Acta, 1853:1658-1671 (2015)), and achieves peak plasma levels of up to 5 μM (Matthew et al., Blood, 122:872 (2013)). The impact of venetoclax on T cells expressing Casp8p41 or reactivating HIV was assessed. In initial experiments, treatment with increasing doses of venetoclax (up to 1 μM) did not result in increased apoptosis of either parental Jurkat cells transfected with no DNA or Jurkat cells expressing eGFP compared to diluent control (FIG. 11A). In contrast, venetoclax (1 μM) significantly increased cell death in Jurkat cells expressing eGFP-Casp8p41 (mean difference 25.7±10.3%, P=0.016, FIG. 11A). This selective enhancement of killing induced by Casp8p41 but not generalized toxicity was further evaluated by treating primary uninfected CD4 T cells with venetoclax. Importantly, uninfected CD4 T cells (N=7 patients) treated with venetoclax (1 μM) for up to five days did not exhibit reduced viability compared to control treated cells (mean difference −7.2±4.8% at Day 5, P=0.16, FIG. 11B). Furthermore, uninfected CD4 T cells treated with venetoclax did not exhibit altered mitogen-induced proliferation as measured by CFSE dilution (mean difference in percent CFSE low cells at Day 3-1.1±7.9%, P=0.90, FIG. 11C). The lack of toxicity of venetoclax towards CD4 T cells from HIV uninfected donors was also seen using cells from HIV infected, but ART suppressed, patients; venetoclax (1 μM) for 24 hours did not adversely affect viability of bulk primary CD4 T cells (mean difference −4.7±1.6% viable, FIG. 11D). These favorable safety findings in vitro have also been seen in vivo: in 40 NHL patients receiving venetoclax monotherapy, only 2 cases of dose limiting neutropenia were observed (Matthew et al., J Clin. Oncol., 32:53 (2014)). These results demonstrate the possibility that venetoclax can be used safely in HIV infected patients.

### Simultaneous Detection of Markers of HIV Infection and Apoptosis

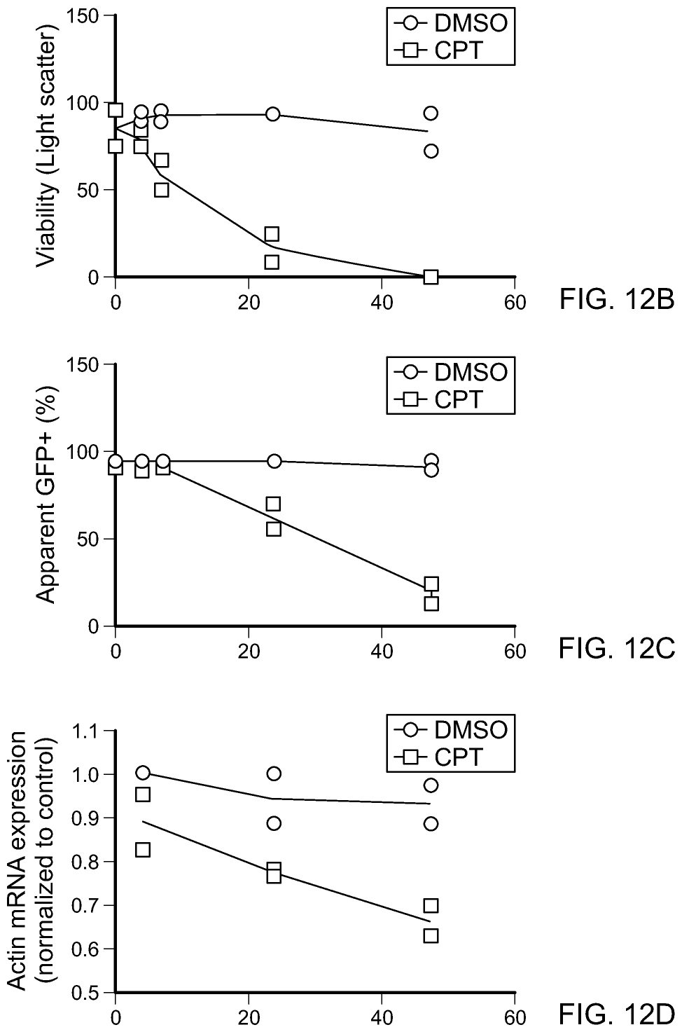

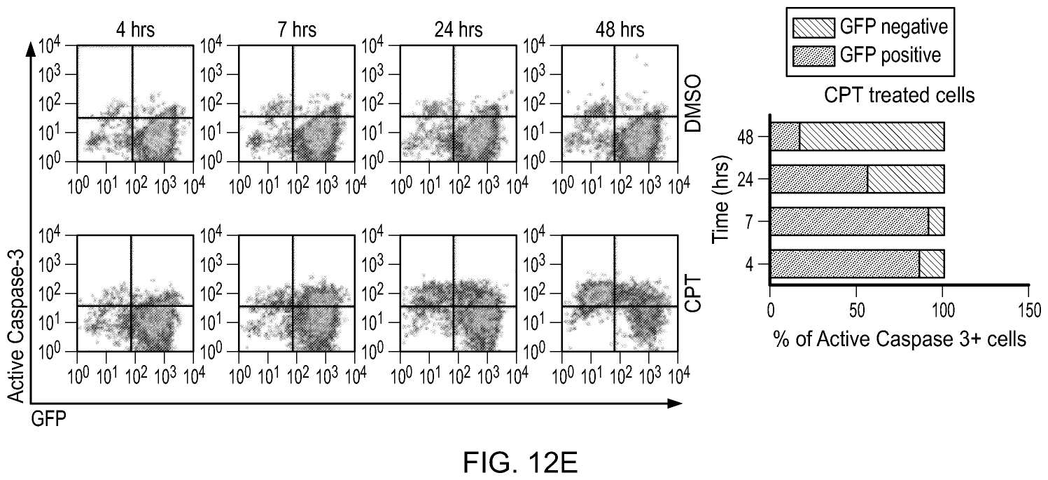

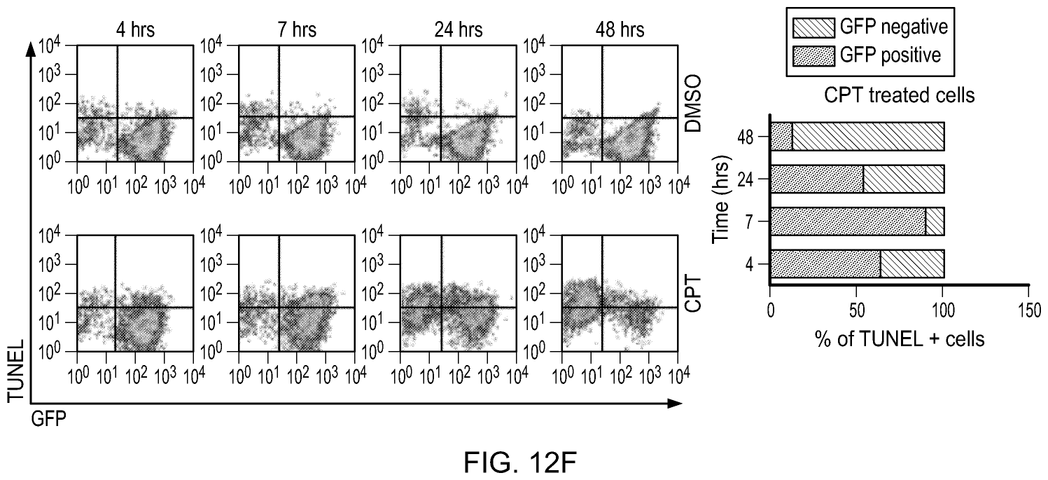

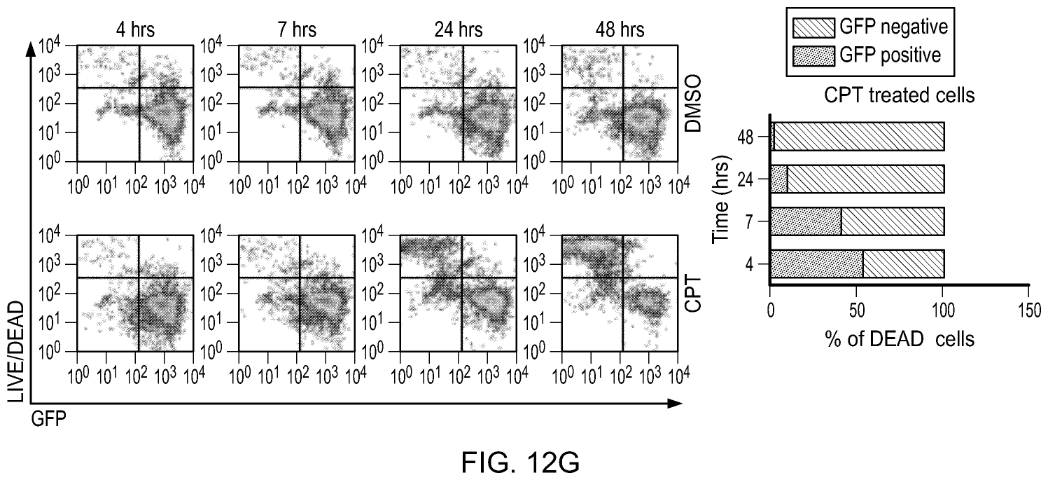

If BCL-2 neutralizes Casp8p41, then antagonizing BCL-2 should augment Casp8p41-induced killing, including when Casp8p41 is generated by latently HIV infected cells that reactivate the virus. Before testing this possibility, whether select markers (protein or nucleic acid based) remain detectable was first assessed when the cell of interest is undergoing death. Jurkat T cells stably expressing eGFP were treated with vehicle control or the cytotoxic quinolone alkaloid CPT to induce apoptosis, then examined for expression of eGFP (as a prototypic cytoplasmic protein) and actin mRNA (as a prototypic mRNA) over time. Treatment of GFP+ Jurkat cells with CPT decreased cell viability as measured by cellular ATP content (FIG. 12A) and light scatter (FIG. 12B) compared to diluent-treated cells.

Coincident with the loss in viability was a decrease in detectable eGFP (17% eGFP+ with CPT vs. 91% eGFP+ with diluent, FIG. 12C) and detectable actin message (34% reduction compared to diluent control) as assessed by qRT-PCR (FIG. 12D). As the proportion of active caspase-3⁺ cells (FIG. 12E), TUNEL+ cells (FIG. 12F) or membrane permeable cells (FIG. 12G) increased over time, there was a reciprocal decrease in detectable eGFP (FIG. 12C) despite the fact that virtually all cells were eGFP positive at baseline. For example, by 48 hours, 97% of CPT treated cells were eGFP negative, despite having been uniformly eGFP positive. Accordingly, estimating the proportion of dead or dying cells that express a degradable marker of interest (e.g., HIV p24 or HIV RNA/DNA) will substantially underestimate the proportion of cells that expressed the marker before encountering the death stimulus. Therefore, the ability to detect protein or nucleic acid based phenotypic markers is impaired as cell death proceeds, rendering this approach insensitive and prone to incorrect phenotyping of the dying cell.

Accordingly, to approach the question of whether HIV reactivation in concert with BCL-2 antagonism reduces cell associated HIV DNA, we compared HIV DNA content in treated compared to control samples rather than simultaneously assessing markers of HIV and cell death in the same cells.

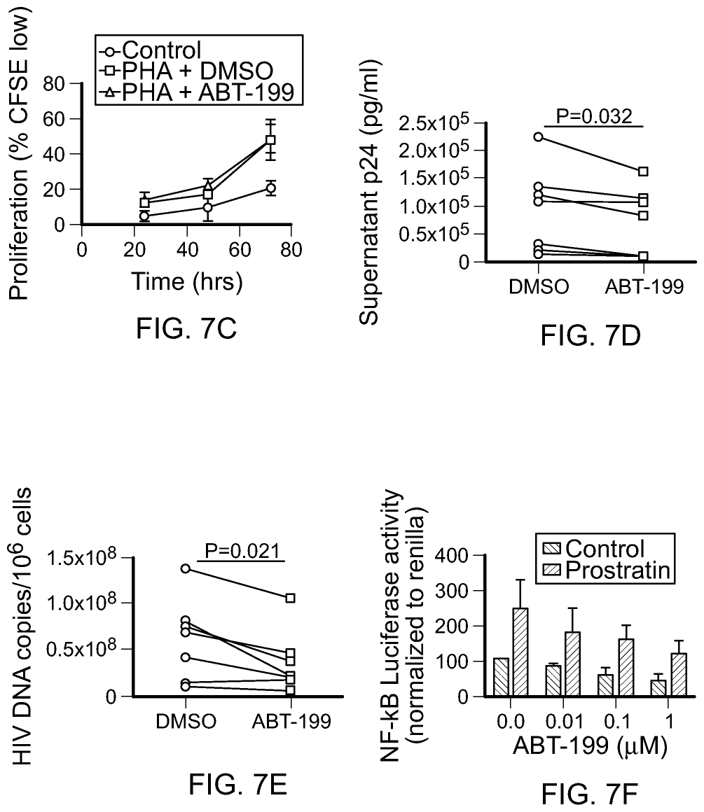

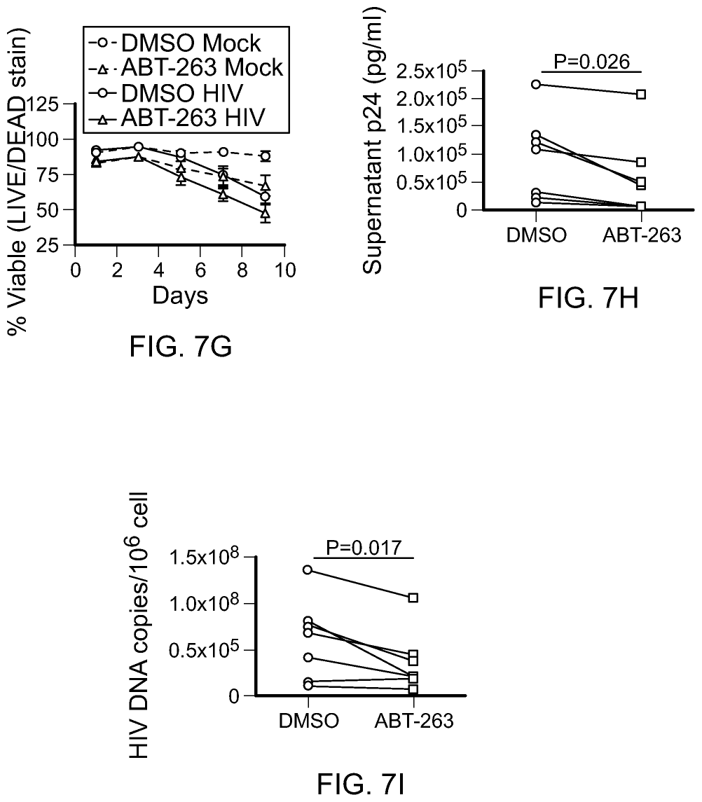

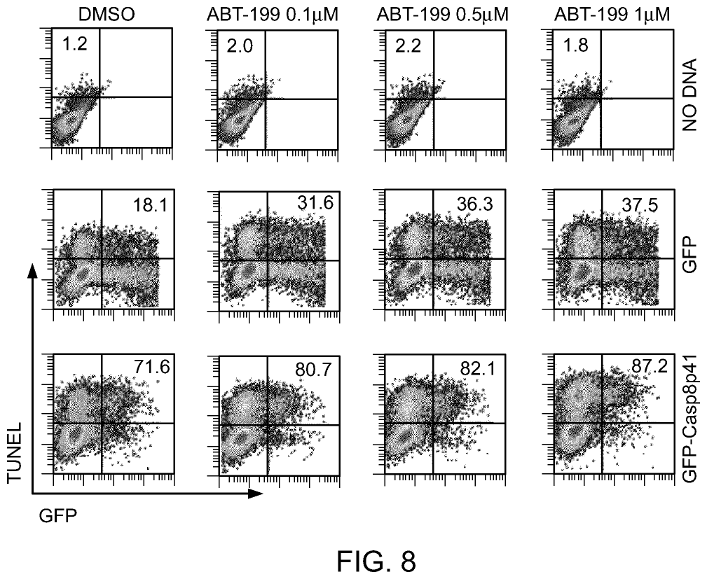

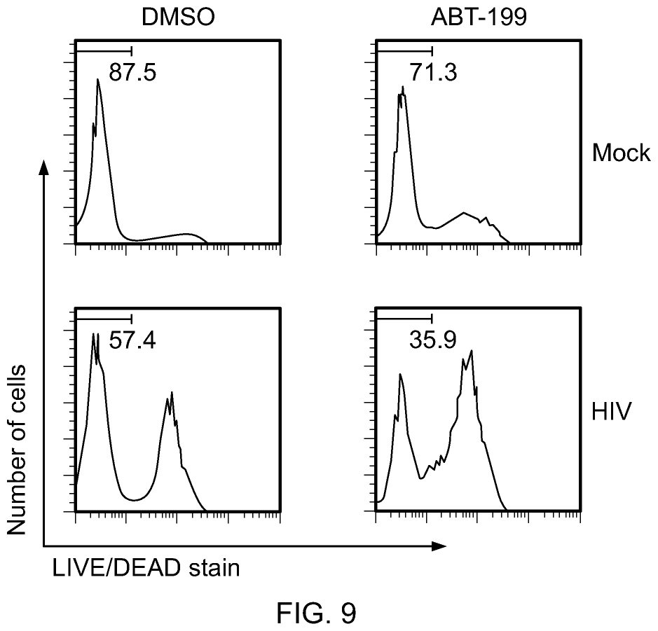

Again, if Bcl-2 inhibits HIV induced cell death by binding the Casp8p41 activator domain, disruption of this interaction should augment both Casp8p41-mediated killing and HIV-induced cell death. Consistent with this possibility, the Bcl-2 antagonist ABT-19914 increased killing by EGFP-Casp8p41 (FIGS. 7A and 8), but not control vector. Likewise, ABT-199 decreased survival of CD4 T cells following acute HIVIIIb infection (FIGS. 7B and 9), but had no effect on survival or mitogen-induced proliferation of mock-infected CD4 T cells (FIG. 7B-C). In addition, ABT-199 reduced supernatant HIV p24 levels (FIG. 7D), potentially reflecting death of HIV producing cells, a direct effect of ABT-199 on HIV replication, or both. Indeed, HIV LTR-Luciferase reporter constructs demonstrated that ABT-199 inhibits both basal and prostratin-induced HIV transcription in primary CD4 T cells (FIG. 7E). While the mechanism by which ABT-199 inhibits HIV LTR transcription is unknown, Bcl-2 overexpression has been associated with NFκB activation in some studies (Mortenson et al., J Cell. Biochem., 102:1171-1179 (2007)), but not others (Grimm et al., J Cell Biol., 134:13-23 (1996)). ABT-199 also decreased cell associated HIV-1 DNA compared to vehicle control treated cells (FIG. 7F). These results, coupled with the lack of toxicity of ABT-199 in uninfected cells (FIG. 7B), demonstrate that ABT-199 preferentially kills HIV-infected cells, resulting in lower HIV replication and fewer HIV infected cells. Similar effects on survival of infected cells, p24 production, and cell-associated HIV-1 DNA were seen with navitoclax (FIG. 7G-I), an inhibitor of Bcl-2, Bcl-xL, and Bcl-w (Tse et al., Cancer Res., 68:3421-3428 (2008)), although navitoclax caused more toxicity in uninfected cells (FIG. 7G).

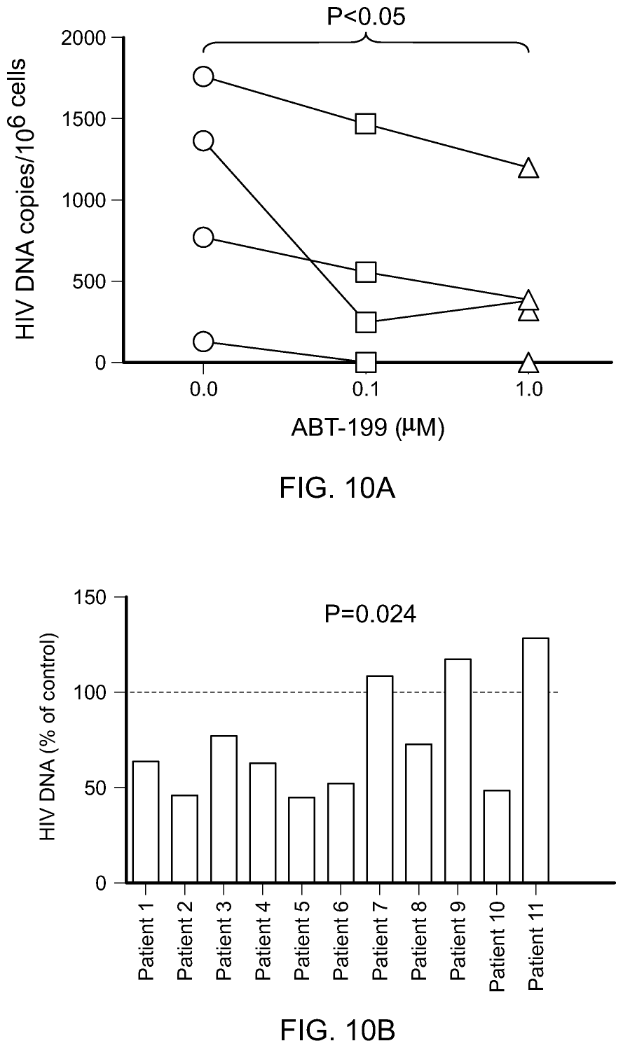

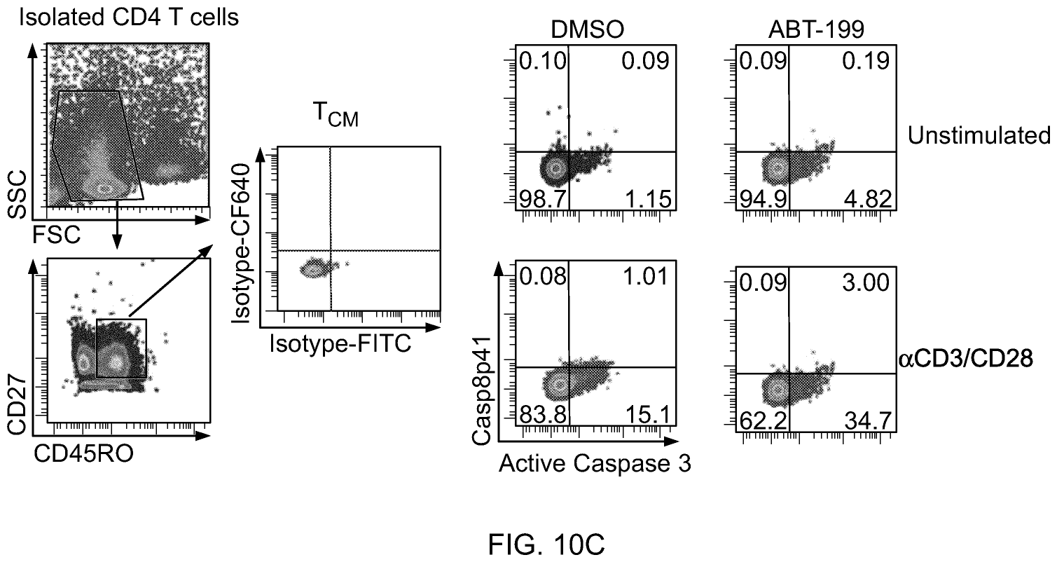

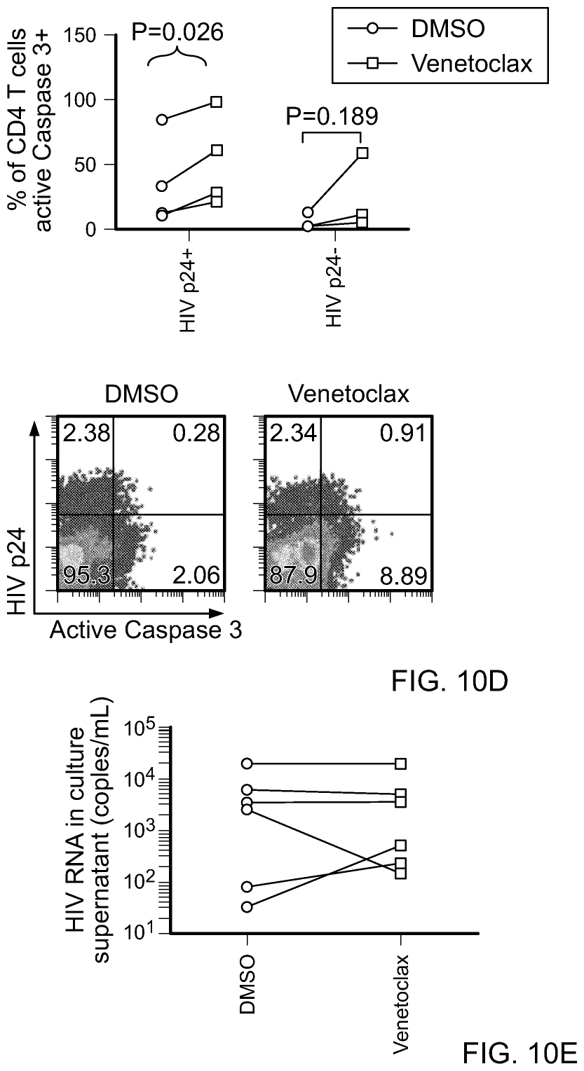

Because ABT-199 reduces HIV replication and decreases the number of cells containing HIV DNA during acute infection in vitro, the effects of this agent during HIV latency were examined. When cryopreserved CD4 T cells from suppressed HIV-infected patients were purified, treated with diluent or ABT-199, and reactivated with αCD3/αCD28 in the presence of tenofovir and raltegravir to prevent spreading infection, ABT-199 reduced cell associated HIV DNA in a dose dependent manner (FIG. 10A). In addition, 8 of 11 (73%) freshly isolated primary CD4 T cell samples with cell associated HIV DNA measurable in the control sample exhibited a decrease after ABT-199 (FIG. 10B). ABT-199 also increased the proportion of Casp8p41 positive T_{CM} cells containing active caspase 3 (FIG. 10C), indicating that Bcl-2 inhibition enhances Casp8p41-mediated killing of HIV-infected T_{CM} cells that reactivate HIV.

Active caspase 3 (a marker of apoptotic cell death) was assessed in p24+(HIV infected) and p24-cells that were primed with venetoclax and reactivated with αCD3/αCD28. Median cell death, as measured by active caspase 3 staining in the p24-cells, was 2.5% in the control treated αCD3/αCD28 sample and was not changed significantly by venetoclax (10.3%, P=0.189, FIG. 10D). In contrast, venetoclax increased the proportion of HIV p24+CD4+ cells that are active caspase 3⁺ compared to control treated cells (from 23% active caspase 3+ in control sample to 47% active caspase 3+ in the venetoclax sample, P=0.026, FIG. 10D). Of note, the frequency of p24 positive cells in reactivated CD4 T cells was similar to other previous reports (Deng et al., Nature, 517:381-385 (2015); and Pegu et al., Nat. Commun., 6:8447 (2015)). HIV RNA in the cell culture supernatant was not altered by venetoclax treatment compared to control (FIG. 10E), indicating that venetoclax does not impair the ability of cells to reactivate HIV.

The results provided herein demonstrate that Casp8p41 interacts with antiapoptotic Bcl-2 family members. Conversely, Bcl-2 inhibition released Casp8p41 and pro-apoptotic Bcl-2 family members, causing more HIV infected cells to die and thereby resulting in reduced HIV replication and fewer cells that contain HIV DNA. Importantly, the HIV reservoir was reported to be stable over time (Josefsson et al., Proc. Natl. Acad. Sci. USA, 110:E4987-4996 (2013)) and unaffected by antiretroviral therapy intensification (Puertas et al., AIDS, 28:325-334 (2014) even in conjunction with therapeutic vaccination (Achenbach et al., The Lancet HIV, 2:e82-e91 (2015)). In contrast, the results provided herein demonstrate that a single treatment with ABT-199 can, through release of Casp8p41, diminish HIV DNA, providing the first evidence that it is possible to pharmacologically diminish HIV reservoir size.

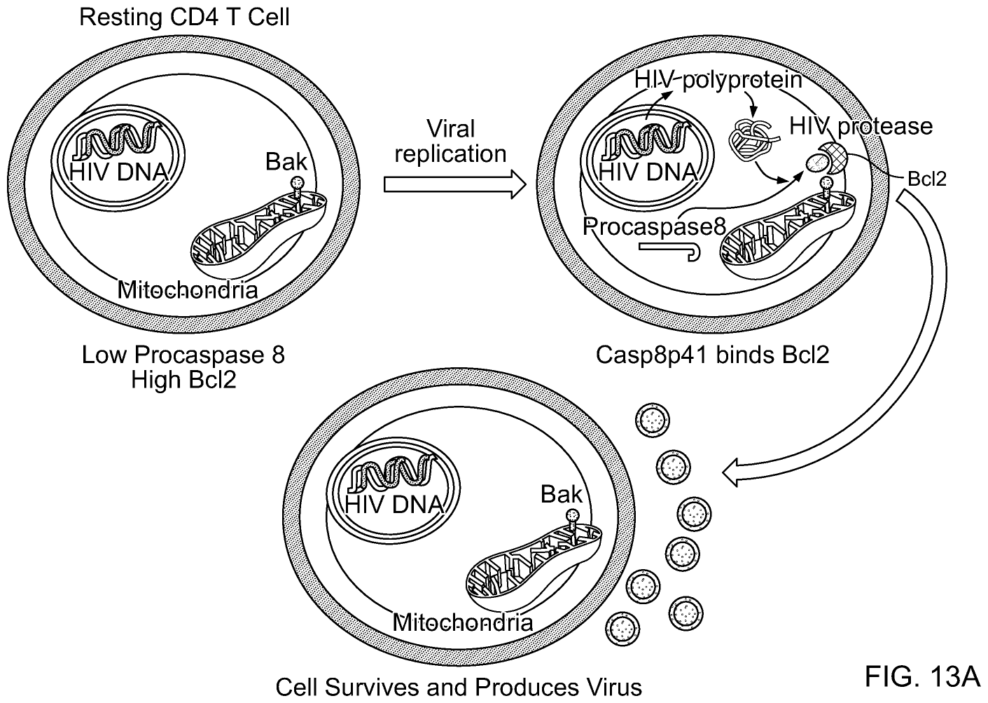

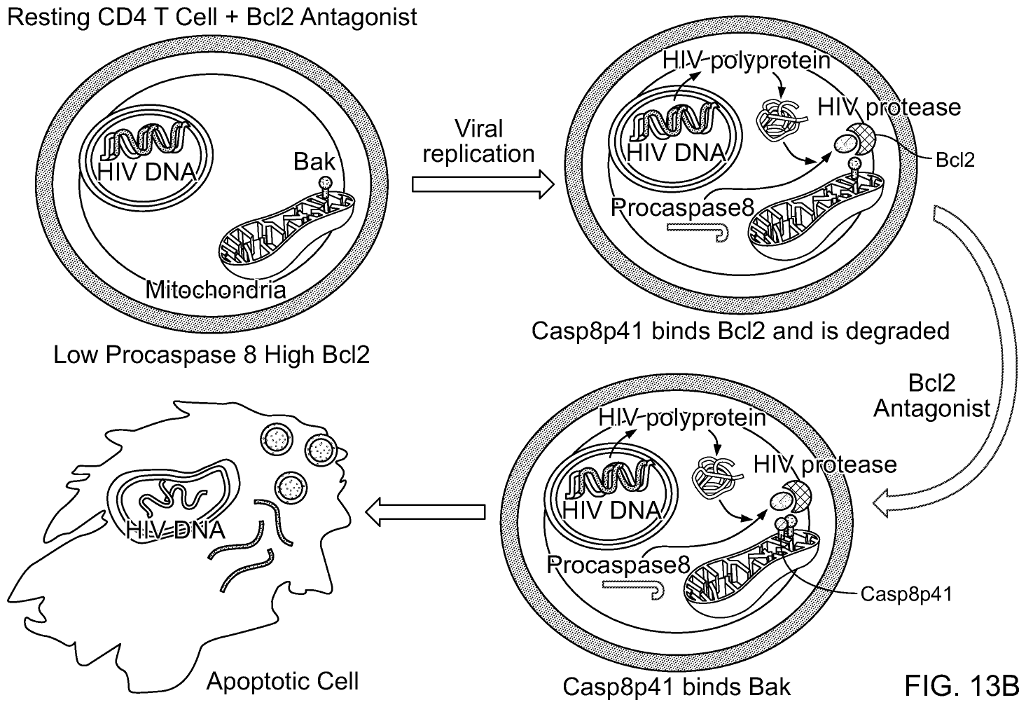

In summary, these results demonstrate that venetoclax-mediated inhibition of BCL-2 in all cells has minimal effect on cell viability in uninfected cells (FIG. 11) yet promotes the preferential killing of p24+459 (HIV replicating) cells. Moreover, these results demonstrate the identification of a clinically relevant treatment that converts HIV reactivation from latency without killing of the reactivating cell (FIG. 13A) into an event that kills the reactivating cell by priming all cells towards apoptosis sensitive phenotype and then allows HIV infected cells, which replicate virus and generate Casp8p41, to undergo apoptosis when BCL-2 is inhibited (FIG. 13B).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for killing HIV infected cells within a human infected with HIV, wherein said method comprises:
(a) administering a Bcl-2 inhibitor to said human, and
(b) administering a latency reversing agent to said human.
